(19) United States
(12) Patent Application Publication
(10) Pub. No.: US 2003/0157072 A1
(43) Pub. Date: Aug. 21, 2003

(54) Nusap, a novel gene encoding a tissue-specific nuclear protein, useful as a diagnostic tool and therapeutic agent

(76) Inventors: Roger Bouillon, Herent (BE); Geert Carmeliet, Oud-Heverlee (BE); Tim Raemaekers, Hasselt (BE)

Correspondence Address: William M Lee Jr, Lee Mann Smith McWilliams Sweeney & Ohlson, PO Box 2786, Chicago, IL, 60690-2786, US

(21) Appl. No.: 10/221,873

(22) PCT Filed: Mar. 16, 2001
(86) PCT No.: PCT/EP01/02971

(51) Int. Cl. C12Q 1/68; A01K 67/027; C07H 21/04; A61K 48/00; A61K 31/59; C12N 9/22; C12P 21/02; C12N 5/06

(52) U.S. Cl. 424/93.21; 800/14; 514/44; 514/167; 536/23.2; 435/6; 435/69.1; 435/199; 435/320.1; 435/325; 530/388.26

(57) ABSTRACT

The invention relates to a eukaryotic nuclear protein NuSAP which is highly expressed in proliferation tissues. More specifically, NuSAP is downregulated in developing bone. NuSAP has a discrete localization in the nucleus. The localization and the expression profile indicate its role in important biological processes such as embryologic development, cell, proliferation, DNA replication and recombination. Its function relates to diseases of the immune system and diseases of the reproductive organs in particular and cancer in general.

### FIELD OF THE INVENTION

This invention relates to a gene which is related to DNA replication, cell proliferation and meiosis and has a discrete localisation in the cell nucleus. This invention further relates to the use of polynucleotide and amino acid sequences in the diagnosis, prevention, and treatment of disorders associated with DNA replication cell proliferation and meiosis, such as cancer, hyperproliferation of cells, disorders of reproduction in eukaryotic cells, disorders of the immune system and disorders related to bones of mammals.

### BACKGROUND OF THE INVENTION

The biologically active form of vitamin D₃, 1α,25-dihydroxyvitamin D₃ [1α,25(OH)₂D₃], has an important role in bone and mineral homeostasis (Bouillon et al. in Endocr. Rev. (1995) 16:200-257). Most actions of 1α,25(OH)₂D₃ are mediated by binding to the vitamin D receptor, a nuclear receptor that functions as a ligand-induced transcription factor that modulates gene expression (Mangelsdorf et al. in Cell (1995) 83:835-839). In vitro experiments have shown that 1α,25(OH)₂D₃ can stimulate or inhibit the differentiation of osteoblasts, the bone-forming cells, depending whether it is given during the proliferation or differentiation phase respectively (Owen et al. in Endocrinology (1991) 128:1496-1504). The pro-differentiating effect of 1α,25(OH)₂D₃ is reflected by the induction of gene expression of osteocalcin, osteopontin and alkaline phosphatase which are characteristic for osteoblast differentiation. Vitamin D responsive elements were identified in several of these proteins (Haussler et al. in J. Bone Miner. Res. (1998) 13:325-349). However, the transcriptional control by 1α,25(OH)₂D₃ is more complex and is likely to be modulated by nuclear matrix- and/or chromatin-factors (Stein et al. in Physiol. Rev. (1996) 76:593-629). In osteoblasts, for example, the vitamin D responsiveness of the osteocalcin gene is altered by interaction with YY1, an ubiquitous transcription factor which localizes predominantly in the nucleolus and also associates with the nuclear matrix (Guo et al. in Proc. Natl. Acad. Sci. USA. (1997) 94:121-126). Moreover, impaired transcriptional control by the glucocorticoid receptor, another steroid receptor, has been observed after deletion of the chromosomal protein HMG1 (Calogero et al. in Nat. Genet. (1999) 22:276-280).

The effect of 1α,25(OH)₂D₃ on the proliferation of osteoblasts and the underlying mechanisms remain however largely unknown. Progression through the cell cycle is in part regulated by the activation and inactivation of cyclin-dependent kinase complexes at specific time points. 1α,25(OH)₂D₃ has the capacity to act as an inhibitor of cell growth of several cancer cells and keratinocytes by blocking the transition from the G1 to the S phase of the cell cycle (Walters in Endocr. Rev. (1992) 13:719-764; Bouillon et al. cited supra). Its coordinate induction of several cyclin-dependent kinase inhibitors, including p21 and p27 either directly or indirectly (Liu et al. in Genes Dev. (1996) 10:142-153; Segaert et al. in J. Invest. Dermatol. (1997) 109:46-54), together with altered cyclin levels (Wang et al. in Cancer Res. (1996) 56:264-247; Verlinden et al. in Mol. Cell. Endocrinol. (1998) 142:57-65) after treatment have been suggested to cooperatively contribute to cell cycle arrest.

Cell proliferation is normally accompanied by the faithful transmission of genetic information. This requires accurate replication and repair of genomic DNA during the S- and G2-phase of the cell cycle. Then, during the mitotic phase, the chromosomes segregate and this involves a coordinated sequence of structural changes including nuclear envelope breakdown, chromosome condensation, collapse of the cytoskeleton and formation of the mitotic spindle (Mc Intosh in Cold Spring Harb. Symp. Quant. Biol. (1991) 56:613-619). It remains unknown until now whether 1α,25(OH)₂D₃ has an effect on the expression of genes involved in one of these processes.

There is a continuous need, with respect to disorders associated with DNA replication, cell proliferation and meiosis, such as cancer, hyperproliferation of cells, disorders of reproduction in eukaryotic cells, disorders of the immune system and disorders related to bones of mammals, to identify and elucidate new proteins having a role in fundamental processes involved in such diseases. In particular, there is a continuous need to identify proteins, the expression level or malfunctioning of which may have a causative role in such diseases, and/or which may be used as diagnostic tools for such diseases. More specifically, since—as described above—vitamin D is an important regulator of bone cell differentiation, a problem addressed by the present invention is the identification of a protein, the expression of which can be modulated by vitamin D supply to osteoblast cells.

### SUMMARY OF THE INVENTION

The present invention is based on the unexpexted discovery of a novel Nuclear Spindle-Associating Protein and the associated polynucleotide and at least some of their relevant biological activities.

The present invention therefore first provides an isolated and purified eukaryotic polynucleotide encoding a Nuclear Spindle-Associating Protein (hereinafter referred as NuSAP) or an intermediate therefor, the said polynucleotide (hereinafter referred as nusap) containing the nucleic acid sequence shown in SEQ ID NO:1 (encoding for mouse NuSAP) or the nucleic acid sequence shown in SEQ ID NO:3 (encoding for human NuSAP), an allelic variant, a homologue, a portion or a mutation thereof, which is able to lead to a functional transcription and/or translation product.

The present invention also includes a protein having the amino acid sequence shown in SEQ ID NO:2 (mouse NuSAP) or the amino acid sequence SEQ ID NO:4 (human NuSAP), a homologue, a variant, a mutation or a portion thereof. This sequence can be encoded by the eukaryotic polynucleotide described above.

The present invention also includes a polynucleotide sequence hybridizing under stringent conditions with the eukaryotic polynucleotide defined above.

The present invention also includes a eukaryotic polynucleotide as defined above, further comprising a polynucleotide which codes for at least a portion of a protein or a marker, the combination of both polynucleotides being arranged in such a way that a fusion protein results after expression.

The present invention further includes a vector, for instance an expression vector or a non-expression vector, comprising at least a eukaryotic polynucleotide as defined above, or a hybridizing sequence thereto.

The present invention also includes a fusion protein resulting after expression of the combination of a eukaryotic polynucleotide as defined above and a further polynucleotide coding for at least a portion of a protein or a marker.

The present invention also includes the use of a eukaryotic polynucleotide or a hybridizing sequence thereto, or a vector (in particular an expression vector) such as defined herein-above for the manufacture of a medicament, in particular for therapeutic treatment or prevention in mammals.

The present invention also includes use of a NuSAP protein or a fusion protein such as defined above, for the manufacture of a medicament for therapeutic treatment or prevention in mammals.

The present invention also includes the use of a eukaryotic polynucleotide or a sequence hybridizing thereto, such as defined herein-above, for the manufacture of a diagnostic tool.

The present invention includes the use of a NuSAP protein, or a fusion protein such as defined above, for the manufacture of a diagnostic tool.

The present invention includes a pharmaceutical composition comprising a eukaryotic polynucleotide or a sequence hybridizing thereto, and a pharmaceutically acceptable carrier.

The present invention further includes a pharmaceutical composition comprising a NuSAP protein or a fusion protein, such as defined herein-above, and a pharmaceutically acceptable carrier.

The present invention also includes a process for the production of a protein NuSAP, comprising culturing a host cell containing an expression vector comprising the polynucleotide EP, and obtaining said protein NuSAP from the cell or the culture medium.

The present invention further includes a transgenic rodent whose genome is heterozygous for an engineered disruption in a rodent NuSAP gene coding for SEQ ID NO:1 or its rodent homologue, wherein said engineered disruption in a heterozygous state inhibits overall or tissue specific production of a NuSAP protein having the amino acid sequence SEQ ID NO:2 or the rodent homologue thereof, thereby resulting in a transgenic rodent which has increased tendency, with respect to a wild-type rodent, to disorders associated with DNA replication, cell proliferation and meiosis, such as cancer, hyperproliferation of cells, disorders of reproduction, disorders of the immune system and disorders related to bones.

The present invention further includes a transgenic rodent whose genome is homozygous for an engineered disruption in a rodent NuSAP gene coding for SEQ ID NO:1 or its rodent homologue, wherein said engineered disruption in a homozygous state inhibits overall or tissue specific production of a NuSAP protein having the amino acid sequence SEQ ID NO:2 or the rodent homologue thereof, thereby resulting in a transgenic rodent which has increased tendency, with respect to a wild-type rodent, to disorders associated with DNA replication, cell proliferation and meiosis, such as cancer, hyperproliferation of cells, disorders of reproduction, disorders of the immune system and disorders related to bones.

The present invention further includes a ribozyme capable of cleaving the mRNA of a NuSAP protein, the said ribozyme including sequences complementary to portions of mRNA obtained from the above-described eukaryotic polynucleotides, for instance obtained from the nucleic acid sequence shown in SEQ ID NO:1 or the nucleic acid sequence shown in SEQ ID NO:3.

The present invention further includes an antisense oligonucleotide capable of blocking expression of an above-described eukaryotic polynucleotide, for instance the nucleic acid sequence shown in SEQ ID NO:1 or the nucleic acid sequence shown in SEQ ID NO:3.

The present invention further includes a vector which produces in a cell, other than a human germ line cell, the above-described ribozyme or antisense oligonucleotide.

The present invention also includes a host cell, other than a human germ line cell, which is transformed with a polynucleotide EP or a sequence hybridizing thereto. The host cell may include a bacterium, fungus, plant or animal cell.

The present invention also provides a method of controlling gene expression of a eukaryotic polynucleotide such as above disclosed, comprising the step of administering to a mammal a pharmaceutical composition comprising at least a therapeutic amount of a vitamin D.

The present invention also provides a method of influencing the biological activity of a Nuclear Spindle-Associating Protein or a fusion protein such as above disclosed, comprising the step of administering to a mammal a pharmaceutical composition comprising at least a therapeutic amount of a vitamin D.

In both above-referred methods, a preferred vitamin D is 1α,25-dihydroxyvitamin D3. Both above-referred methods are preferably for modulating the process of DNA replication or DNA recombination in the said mammal.

The present invention further provides an antibody, preferably a monoclonal antibody, being able to discriminate between a NuSAP Protein with and without a post-translationally modified amino-acid.

The present invention also provides a method of prevention or treatment of a disorder associated with DNA replication, cell proliferation or meiosis, comprising administering to a mammal suffering from such disorder a therapeutically effective amount of a eukaryotic polynucleotide such above described. The said disorder may be in particular selected from cancer, hyperproliferation of cells, disorders of reproduction in eukaryotic cells, disorders of the immune system, disorders related to bones of mammals and disorders caused by mutations in nuclear structural proteins such as the Emery-Dreifuss muscular distrophy, the dilated cardiomyopathy and lipodistrophy.

### DESCRIPTION OF THE DRAWINGS

FIG. 1 shows the identification of a novel 1,25(OH)₂D₃ regulated gene in proliferating mouse osteoblastic MC3T3-E1 cells. (a) The proliferation of MC3T3-E1 cells was analyzed after treatment for 24 and 48 h with varying doses of 1,25(OH)₂D₃ by monitoring [³H]-thymidine incorporation. Data represent mean±SD of six replicates of a representative experiment. Values of vehicle (ethanol) treated cells were taken as 100%. (*P<0.001; versus vehicle, t test)

FIG. 2 represents a Northern blot analysis showing nusap gene expression in sub-confluent cultures of MC3T3-E1 cells after treatment with vehicle or 1,25(OH)₂D₃ (10⁻⁸ M) for 24 hours. The size (kb) of the three transcripts is indicated.

FIG. 3 represents a Northern blot analysis showing nusap gene expression relative to growth markers (histone H4) and differentiation markers (osteocalcin) in developing MC3T3-E1 cells. Cells were cultured for 4 weeks during which they proceeded through subsequent stages of development: proliferation (I), matrix maturation (II), and mineralization (III). The regulation of gene expression of the different stage-specific markers by 1,25(OH)₂D₃ (10⁻⁸ M for 24 hours) is also shown. Subsequent hybridizations were performed with the following probes: human histone H4, pFO002; mouse osteocalcin, p923; and a 0.8 kb NuSAP BglII/AspI restriction fragment. The 18S cDNA probe was used as a control to assess equal loading.

FIG. 4 shows the deduced amino acid sequence of mouse and human NuSAP and its alignment with predicted proteins from other species (A), and with the SAP motif consensus sequence (B). (A) Identical and similar residues are shaded in black. Sub-groups of homologous residues were defined as follows: positively charged residues (R,K), negatively charged residues (E,D), and hydrophobic residues (L,V,I,F,M). Gaps, indicated by dashes or numbers between parenthesis, were introduced for optimal alignment. Boxed at the N-terminus is the potential SAP motif, and at the C-terminus (in dashed lines) is a conserved stretch of charged residues that may be important for electrostatic interactions. Underlined is the potential nuclear localization signal identified in the mouse sequence. (B) Residues within the SAP motif consensus sequence have been defined by Aravind et al. in Trends Biochem. Sci. (2000) 25:112-4 as h (hydrophobic), p (polar), l (aliphatic), and b (bulky). Also shown is the sequence of mouse AcinusL (Accession No. AAF89661), a SAP module containing protein. Shaded in black are residues which agree with the consensus sequence, and in grey are residues which conform to the similarity as described in (A). Sequences besides those of mouse and human, were deduced from expressed sequence tags. The Genbank TM accession numbers are as follows: Hs, Homo sapiens (human) (AAG25874); Bt, Bos taurus (cattle) (BE480183); Mm, Mus musculus (mouse) (AAG31285); Rn, Rattus norvegicus (rat) (AA923940); Gg, Gallus gallus (chicken) (AJ392813); Xl, Xenopus laevis (clawed frog) (AW642384); and Dr, Danio renio (zebrafish) (AI545826, AI958745).

FIG. 5 shows expression of the nusap gene in sub-confluent MC3T3-E1 cell cultures after serum withdrawal. Northern blot analysis of nusap expression after serum withdrawal (0.1% fetal calf serum FCS) and upon serum re-addition (10% FCS). Cells were harvested at the indicated time points (hours). The different cell cycle phases were assigned based on the RNA levels of histone H4, a S-phase marker. The blots were hybridized with the following cDNA probes: human histone H4, pFO002; and a 0.8 kb NuSAP BglII/AspI restriction fragment. The 18S cDNA probe was used as a control to assess equal loading.

FIG. 6 shows expression of the nusap gene in sub-confluent MC3T3-E1 cell cultures after hydroxyurea treatment. Northern blot analysis of nusap expression in hydroxyurea treated (1 mM) MC3T3-E1 cells. Cells were harvested at the indicated time points (minutes) after treatment. The different cell cycle phases were assigned based on the RNA levels of histone H4, a S-phase marker. The blots were hybridized with the following cDNA probes: human histone H4, pFO002; and a 0.8 kb NuSAP BglII/AspI restriction fragment. The 18S cDNA probe was used as a control to assess equal loading.

FIG. 7 shows expression of the nusap gene in synchronised sub-confluent MC3T3-E1 cell cultures. Northern blot analysis of nusap expression in MC3T3-E1 cells synchronized using a double thymidine block. Cells were washed and released in growth medium and harvested at the indicated time points (hours). The different cell cycle phases were assigned based on the RNA levels of histone H4, a S-phase marker. The blots were hybridized with the following cDNA probes: human histone H4, pFO002; and a 0.8 kb NuSAP BglII/AspI restriction fragment. The 18S cDNA probe was used as a control to assess equal loading.

FIG. 8 shows expression of nusap in mouse (C57BL/6) tissues and embryo. (a) Northern blot analysis of nusap expression in tissues from 2 months old mice. (b) Northern blot analysis of nusap expression in whole embryo's from different developmental stages (days post-coitum). The following cDNA probes were used: human histone H4 (pFO002), and a 0.8 kb NuSAP BglII/AspI restriction fragment. To assess equal loading 18S, β-actin, and glyceraldehyde 3-phosphate dehydrogenase (GAPDH) cDNA probes were used.

FIG. 9 shows in situ hybridization of nusap in mouse tissues (C57BL/6). Sections were hybridized with a DIG-labeled nusap probe. (a) In sections of the adult testis, the primary spermatocytes (arrowhead) were stained positively. Spermatogonia, denoted by an arrow, were negative. (b) Expression of nusap in adult ovary sections was strong in oocytes (star). (c) In sections of the adult thymus, the subcapsular outer cortex region (arrows) showed positive staining. (d) Expression of nusap RNA in sections of the small intestine of a 19 days post-coitum embryo was intense in cells in the crypts of the villi (arrow). Bars: (a, d) 50 μm; (b, c) 30 μm.

FIG. 10 shows expression of nusap in tumor tissues. Elevated expression levels of NuSAP in cDNA pools of human tumor tissues (designated as T) is compared to the expression of NuSAP in cDNA pools obtained from the same tissue from healthy individuals (designated as N). Quantitive detecture of nusap is detected by exposure on a phosphorimager.

FIG. 11 shows that NuSAP localizes to the nucleolus and to intranuclear filaments. Indirect immunofluorescence analysis of transfected COS-1 cells by confocal microscopy, shows GFP-tagged NuSAP localizing to the nucleus and to the nucleolus (A, D)(Green) which is stained for nucleolin (B)(red) respectively. C is the merger of A and B. Interaction between NuSAP-GFP and nucleolin was shown by immunoprecipitating COS-1 cell lysates (L) with antibodies to GFP (IPg) and to nucleolin (IPn) followed by Western blot analysis with antibodies to nucleolin and GFP (E). Consecutive sections taken at 1 μm z-axis increments show that GFP-tagged NuSAP localizes to an intranuclear filamentous network at the nuclear periphery of transfected COS-1 cells (F and G). At the core of the nucleus a more diffuse fluorescence pattern is observed (H). (Bar: 5 μm)

FIG. 12 shows that transfected NuSAP localizes to sites of DNA replication in S-phase cells. COS-1 cells were transiently transfected with GFP-tagged NuSAP and incubated in medium containing BrdU (10 μM) for 30 minutes before fixation in 70% ethanol. Indirect immunofluorescence analysis of transfected COS-1 cells in S-phase shows NuSAP-GFP foci (A) lying adjacent to sites of BrdUrd incorporation (B) (red). C is the merger of A and B. In some cells, BrdUrd incorporation is distinctly aggregated (E), and these sites overlap with NuSAP-GFP (D) and F (which is the merger of D and E).

FIG. 13 shows that during mitosis transfected NuSAP localizes to the chromosomes and microtubules. NuSAP expressing cells were examined for GFP fluorescence (green) and indirect immunofluorescence labeling of α-tubulin (red). DNA was stained with Hoechst 33258 (blue). (A) Assignment of the different stages of mitosis was based on morphological features. Fluorescence derived from GFP was merged with that derived from the DNA stain (middle merger) or from anti-α-tubulin staining (bottom merger). Bar: 15 μm.

FIG. 14 shows the localization of transfected NuSAP after treatment with nocodazole and taxol. Cells in mitosis were either treated with nocodazole for 1 hour (10 μM), or treated similarly and then washed and released in growth medium for 1 hour. Taxol treatment (5 μM for 1 hour) induced the formation of microtubule asters. NuSAP-expressing cells were examined for GFP fluorescence (green) and indirect immunofluorescence labeling of α-tubulin (red). Merge is an overlay oftubilin and NuSAP-GFP. DNA is detected by Hoechst staining (blue). Bar: 10 μm.

FIG. 15 shows the overexpression of sense and antisense NuSAP in COS-1 cells. COS-1 cells were transiently transfected with sense and antisense constructs of NuSAP. As a control, a vector without insert was used. Cell proliferation was analyzed 48 hours post-transfection and monitored by [³H]-thymidine incorporation. Data represent mean±standard deviation of six replicates of a representative experiment. (**P<0.01, *P<0.05; versus control, t test).

FIG. 16 shows overexpression of sense and antisense NuSAP in NIH3T3 cells. NIH3T3 cells were transiently transfected with sense and antisense constructs of NuSAP. As a control, a vector without insert was used. Cell proliferation was analyzed 48 hours post-transfection and monitored by [³H]-thymidine incorporation. Data represent mean±standard deviation of six replicates of a representative experiment. (**P<0.01, *P<0.05; versus control, t test).

FIG. 17 represents cell cycle profile of COS-1 cells 48 hours post transfection, showing a clear increase of S-phase population in cells overexpressing GFP-tagged NuSAP, relative to the control (GFP-vector).

### DEFINITIONS

The term “hybridization”, as used herein, refers to any process by which a strand of nucleic acid binds with a complementary strand through base pairing.

The term “complementary”, as used herein, refers to the natural binding of polynucleotides under permissive salt and temperature conditions by base pairing. For example, the sequence 5′-AGTT-3′ binds to the complementary sequence 5′-AACT-3′. Complementarity between two single-stranded molecules may be “partial”, in which only some of the nucleic acids bind, or it may be “complete” when total complementarity exists between the single stranded molecules. The degree of complementarity between polynucleotides has significant effects on the efficiency and strength of hybridization between polynucleotides. This is of particular importance in amplification reactions, which depend upon binding between polynucleotides.

The term “homology” or “homologue”, as used herein, refers to a degree of similarity between two sequences.

The term “stringent conditions”, as used herein, is the stringency which occurs within a range from about Tm −5° C. (5° C. below the melting temperature of the probe) to about 20° C. to 25° C. below Tm. As will be understood by those skilled in the art, the stringency of hybridization may be altered to identify or detect identical or related polynucleotide sequences.

The term “antisense”, as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) or interest in a reverse orientation to a promoter which permits the synthesis of a complementary strand. Once introduced into a cell, this transcribed strand combines with natural sequences produced by the cell to form duplexes. These duplexes then block either the further transcription or translation. In this manner, mutant phenotypes may be generated.

The term “portion”, as used herein, for instance with regard to a protein, refers to fragments thereof. The protein fragments may range in size from at least about 10 amino acid residues, preferably at least about 20 amino acid residues and more preferably at least about 40 amino acid residues, up to the entire amino acid sequence except for one amino acid. The same definition applies to nucleotide sequences as well, the fragments thereof may range in size from at least about 15 nucleotides, preferably at least about 30 nucleotides and more preferably at least about 60 nucleotides.

“Transformation”, as defined herein, describes a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such transformed cells include stably transformed cells in which the inserted DNA is capable of replication either as an autonomously replicating plasmid or as part of the host chromosome. They also include cells which transiently express the inserted DNA or RNA for limited periods of time.

As used herein, an “allele” or “allelic” sequence is an alternative form of the gene which may result from at least one mutation in the polynucleotide. Alleles may result in altered mRNAs or polypeptides whose structure or function may or may not be altered.

The term “intermediate”, as used herein with respect to a NuSAP protein, means a messenger RNA, a sense RNA or an anti-sense RNA which eventually can lead to a Nuclear Spindle-Associating Protein, a homologue, a variant, a mutation or a portion thereof.

As used therein, the term “vitamin D” is defined according to the nomenclature of Vitamin D established by the International Union of Pure and Applied Chemistry and includes all vitamin D compounds and derivatives mentioned therein.

### DETAILED DESCRIPTION OF THE INVENTION

The present invention is based on the discovery of a new nuclear protein (NuSAP) involved in cell proliferation, eukaryotic polynucleotides encoding NuSAP and their use for the diagnosis, prevention and treatment of various diseases including various forms of cancer, hyperproliferation of cells and other disorders associated with cell proliferation and meiosis.

The present invention discloses eukaryotic polynucleotides encoding for NuSAP. The human (SEQ ID NO:3) and mouse (SEQ ID NO:1) polynucleotides of the novel gene nusap have been cloned. The chromosome localisation of the human nusap gene is on 15q15. The human and mouse sequences are more than 60% identical at the amino acid level (SEQ ID NO:4 and SEQ ID NO:2 respectively).

Homologues of nusap are present in organisms other than mouse and man. It will be appreciated by those skilled in the art that sequences originating from these other organisms will differ to some extent from the mouse and human nucleic acid sequences specifically disclosed herein.

Also, it will be appreciated by those skilled in the art that, as a result of the degeneracy of the genetic code, a multitude of nucleotide sequences encoding NuSAP, some bearing low similarity to the nucleotide sequences of any known and naturally occurring gene, may be produced. Thus, the invention contemplates each possible variation and/or portion of nucleotide sequence that could be made by selecting combinations based on possible codon choices.

The invention also encompasses production of polynucleotide sequences, or portions thereof, which encode NuSAP and its derivatives, entirely by synthetic chemistry. Synthetic sequences may be used to introduce mutations into a sequence encoding NuSAP or any portion thereof. Such sequences may also be inserted in any available expression vector and cell system, while using reagents well known in the.

Altered polynucleotides encoding NuSAP that are encompassed in the invention include deletions, insertions, or substitutions of different nucleotides resulting in a polynucleotide encoding a portion and/or a mutated version of NuSAP.

The polynucleotides encoding NuSAP may be extended by using a portion of the nucleotide sequence and employing various methods known in the art to detect upstream sequences such as promoters and regulatory elements. Such methods include among others “restriction-site” polymerase chain reaction (hereinafter referred as PCR), inverse PCR and capture PCR or chromosome walking techniques.

The invention encompasses NuSAP but also NuSAP variants which retain the biological activity of NuSAP. A preferred NuSAP variant is one having at least 80%, more preferably at least 90%, amino acid sequence identity to the NuSAP amino acid sequence. The encoded protein may also contain deletions, insertions, or substitutions of amino acid residues which produce a silent mutation and result in a biologically equivalent of NuSAP.

Also encompassed by the present invention are polynucleotide sequences that are capable of hybridizing to the claimed polynucleotide sequences, in particular those shown in SEQ ID NO:1 and SEQ ID NO:3, under various stringent conditions.

In another embodiment of the present invention, polynucleotide sequences or portions thereof which encode NuSAP, or fusion proteins or biological equivalents thereof, may be used in recombinant DNA molecules to direct expression of NuSAP in appropriate host cells. As will be understood by those skilled in the art, it may be advantageous to produce NuSAP-encoding polynucleotides possessing non-naturally occurring codons, for instance in order to increase the half-life of the transcript. The nucleotide sequences of the present invention can farther be engineered using methods generally known in the art to modify the cloning, processing and/or expression of the gene product. Chimeric fusion proteins can be engineered to tag the protein an/or to enhance purification. A fusion protein in the latter case may be engineered to contain a cleavage site located between the NuSAP encoding sequence and the heterologous protein sequence.

In order to express biologically active NuSAP, the polynucleotide encoding NuSAP or a biological equivalent thereof may be inserted into an appropriate vector, i.e. a vector which contains the necessary elements for the transcription and translation of the inserted coding sequence. Methods used to construct such expression vectors are well known to those skilled in the art. Expression vectors derived from retroviruses, adenoviruses, herpes or vaccinia viruses or from various plasmids may be used for delivering the polynucleotides of this invention to the targeted organ tissue or cell population. Specific examples of such vectors include the pcDNA3.1(−)/Myc-His B plasmid, the pEGFP-N1 plasmid and the like. In order to express biologically active NuSAP, integration of the fused gene into the chromosomal DNA of a host may be considered as well.

A variety of expression vector/host systems may be utilized to contain and express sequences encoding NuSAP. Host cells can include, but are not limited to bacteria, yeast, insect, plant or animal cells. A host cell strain may be chosen to modulate the expressed protein in the desired fashion. For long-term, high-yield production of recombinant proteins, stable expression is preferred. Any number of selection system may be used to recover transformed cells. Alternatively, host cells which contain the polynucleotide encoding NuSAP and expressing NuSAP may be identified by a variety of procedures known to those skilled in the art, such as labels and conjugation techniques typically used in various nucleic acid and amino acid assays.

Host cells transformed with polynucleotides encoding NuSAP may be cultured under conditions suitable for the expression and recovery of the protein from the cell culture. The protein produced by a recombinant cell may be secreted or contained intracellularly depending on the sequence and/or the vector used. Expression vectors containing polynucleotides encoding NuSAP may be designed to contain signal sequences which direct secretion of NuSAP through a prokaryotic or eukaryotic membrane. Other recombinant constructions may be used to join sequences encoding NuSAP to polynucleotides encoding a polypeptide domain which will facilitate purification of soluble proteins. Such purification facilitating domains are well known in the art.

In one embodiment of the present invention it is shown that over-expression of antisense NuSAP inhibits cell proliferation. Antisense strands may be produced by any method, including synthesis by ligating the sequence of interest in a reverse orientation in an expression vector, which permits the synthesis of the complementary strand. Antisense molecules may be used to modulate NuSAP activity or to achieve regulation of the gene function. The disclosed polynucleotides encoding for NuSAP antisense strands, or a vector containing these sequences may for instance be administered to a mammal in order to prevent or treat a disorder associated with hyperproliferation of cells. Such disorders include various types of cancer. A composition comprising therapeutically effective amounts of antisense strands to a polynucleotide (RNA) encoding NuSAP may be mixed with any pharmaceutically acceptable carrier. The invention thus provides an antisense nucleic acid molecule that is complementary to at least a portion of the mRNA encoding a NuSAP protein. Antisense nucleic acid molecules can be RNA or single-stranded DNA, and can be complementary to the entire mRNA molecule encoding NuSAP or to only a portion thereof. These antisense molecules can be used to reduce levels of NuSAP, for instance by introducing into cells an RNA or single-stranded DNA molecule that is complementary to at least a portion of the mRNA of NuSAP (i.e. by introducing an antisense molecule). For a general discussion of antisense molecules and their use, reference is made to Rossi Br Med. Bull. (1995) 51:217-25.

The invention further provides a special category of antisense RNA molecules, known as ribozymes, having recognition sequences complementary to specific regions of the mRNA encoding NuSAP. Ribozymes not only complex with target sequences via complementary antisense sequences but also catalyze the hydrolysis, or cleavage, of the template mRNA molecule.

Expression of a ribozyme in a cell can inhibit gene expression. More particularly, a ribozyme having a recognition sequence complementary to a region of a mRNA encoding NuSAP can be used to decrease expression of NuSAP. A vector may be used for introduction of the ribozyme into a cell. For a general discussion of ribozymes and their use, reference is made to Christoffersen. J. Med. Chem. (1995) 38:2023-37.

A possible alternative comprises the use of antibodies raised against NuSAP. A method for obtaining for instance mouse anti-human monoclonal antibodies is well known to those skilled in the art, including the humanization of antibodies by replacing amino acids in the non-antigen binding regions in order to more closely resemble a human antibody, while still retaining the original binding ability. Such a method is described for instance in Fundamental Immunology (1999), Lippincott-Raven. Protein biological activity is often regulated by post-translational modifications (hereinafter referred as PTM). Antibodies raised against NuSAP that can discriminate against a particular PTM (e.g. phosphorylation) are a useful tool in the diagnosis of a disease caused by a modified pattern compared to healthy individuals, independent from the fact whether the modified PTM is caused by a mutation in the protein itself or due to a mutation leading to a decreased function.

Pharmaceutically acceptable carriers as used throughout this application are well known to those skilled in the art and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals. The pharmaceutical compositions of the present invention may be prepared in any known manner, for instance by homogeneously mixing, coating and/or grinding the active ingredients, in a one-step or multi-steps procedure, with the selected carrier material and, where appropriate, the other additives such as surface-active agents. may also be prepared by micronisation, for instance in view to obtain them in the form of microspheres usually having a diameter of about 1 to 10 μm, namely for the manufacture of microcapsules for controlled or sustained release of the active ingredients. Additional ingredients may be included in order to control the duration of action of the active ingredient in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethylcellulose, polymethyl methacrylate and the other above-described polymers. Such methods include colloid drug delivery systems like liposomes, microspheres, microemulsions, nanoparticles, nanocapsules and so on. Depending on the route of administration, the pharmaceutical composition may require protective coatings.

The pharmaceutical compositions of the invention may be administered by any route well known in the art, i.e. orally, intranasally, subcutaneously, intramuscularly, intradermally, intravenously, intra-arterially, parenterally or by catheterization.

Over-expression of sense NuSAP promotes cell proliferation and in particular promotes cell proliferation via DNA replication. Therefore the above-disclosed eukaryotic polynucleotides encoding for NuSAP, including portions thereof, may usefully be administered to a mammal in order to prevent or treat a disorder associated with decreased cell proliferation.

The NuSAP protein and the nusap gene can be used as a diagnostic tool in diseases were the level of DNA synthesis is disturbed, such as specific types of cancer, or in diseases that are caused by an impaired recombination. Differences in NuSAP concentration and/or amounts can be detected at the protein level by Western blots, or at the mRNA level by Northern blots or quantitative RT-PCR. As can be seen in one of the following examples, the elevated expression level of nusap differs depending from the cancer being considered. At the genomic level, mutations, deletions or translocations of NuSAP can be detected by hybridisation with NuSAP oligo- or polynucleotide probes or by detection procedures in which a PCR with nusap primers is performed.

NuSAP also relates to diseases caused by genetic defects of structural nuclear proteins. The following examples show that NuSAP is a constituent of a fibrous structure in the nucleus, the nuclear matrix. Diseases considered here are namely the Emery-Dreifuss muscular dystrophy caused by deletions or mutations in the Emerin gene or the lamin A gene (Bione et al. in Nature Genet. (1994) 8:323-7; Bonne et al. in Nature Genet. (1999) 21:285-8) and the dilated cardiomyopathy and lipodystrophy being caused by mutations in the lamin A gene (Cao et al. in Hum. Molec. Genet. (2000) 9: 109-112; Fatkin et al. in New Eng. J. Med. (1999) 341: 1715-1724).

In one embodiment of the invention the tissue specific expression of nusap is demonstrated. Nusap gene expression is for instance strong in meiotic cells of the reproductive organs. Therefore, the disclosed polynucleotides encoding for NuSAP, a vector containing these sequences, NuSAP or a portion thereof may be usefully administered to a mammal in order to prevent or treat a reproductive disorder. Nusap gene expression is further strong in mitogenic cells of the immune system and in bone cells. Therefore, the disclosed polynucleotides encoding NuSAP, the NuSAP protein or a portion thereof may be usefully administered to a mammal in order to prevent or treat a disorder associated with the immune system or a disorder related to bone, in particular to bone growth.

In another embodiment of the present invention, the anti-proliferative effect of 1α,25-dihydroxyvitamin D3, through downregulation of nusap expression is demonstrated. 1α,25-dihydroxyvitamin D3 may thus be used to modulate nusap gene expression, controlling thereby or interfering with cell regulatory processes like DNA replication, DNA repair and recombination. Therefore, a therapeutically effective amount of vitamin D3 may be administered to a mammal, more preferably a human being, in order to prevent or treat a disorder associated with cell hyperproliferation.

Any of the diagnostic and therapeutic methods described herein-above may be applied to any mammal in need of such therapy or diagnosis, for example dogs, cats, cows, horses, rabbits, monkeys, and most preferably, humans.

As previously indicated, NuSAP plays a role in DNA replication and recombination. Involvement in DNA replication is suggested by colocalization to sites of bromodeoxyuridine (hereinafter referred as BrDU) incorporation (DNA replication foci) and by a correlation with histone H4 expression (marker linked to DNA replication); upregulation of nusap expression during the replicative phase of the cell cycle (S-phase); restriction of nusap expression to actively dividing mitogenic or meiotic cells. Another biological function of NuSAP in DNA recombination derives from the fact that tissue-specific nusap expression does not always follow histone H4 expression. In the adult testis, high expression was for instance confined to the primary spermatocytes in the late pachytene stage of meiosis, during which recombination is completed. In lymphoid tissues recombination occurs during immunoglobulin and T cell receptor gene arrangements and/or immunoglobulin class switching. Therefore, the disclosed polynucleotides encoding for NuSAP, a vector containing these sequences, the NuSAP protein or a portion thereof can be used in recombination and DNA replication technology, for instance for gene transfer and/or ectopic expression.

Various assays, well known in the art, may be used to detect disorders associated with nusap expression. Diagnostic tools may comprise antibodies raised against the NuSAP protein in its broad meaning. Such antibodies may be used for the diagnosis of conditions or diseases characterized by expression of NuSAP, or in assays to monitor patients being treated with NuSAP or its encoding polynucleotides. Monitoring protocols include for instance ELISA, radio-immune assays and fluorescence-activated cell-sorter analysis (hereinafter referred as FACS) and are well known in the art.

Polynucleotides encoding NuSAP or a portion thereof may also be used for diagnostic purposes. The polynucleotides which may be used include oligonucleotide sequences; antisense RNA and DNA molecules. They may be used to detect and quantify gene expression in biopsied tissues in which expression of NuSAP may be correlated with a disease such as above defined. The diagnostic assay may be designed and used to distinguish between the absence, the presence and the excessive expression of NuSAP, and to monitor regulation of NuSAP levels during therapeutic intervention.

In one aspect, hybridization with PCR probes which are capable of detecting polynucleotide sequences, including genomic sequences, encoding NuSAP or closely related molecules, may be used to identify polynucleotide sequences encoding NuSAP. The specificity of the probe, whether it is made from a highly specific region, e.g. 10 unique nucleotides in the 5′ regulatory region, or a less specific region, e.g., especially in the 3′ coding region, and the stringency of the hybridization or amplification will determine whether the probe identifies only naturally occurring sequences encoding NuSAP, alleles, or related sequences. Probes used for the detection of related sequences, should preferably contain at least about 50% of the nucleotides from any of the NuSAP encoding sequences. Means for producing specific hybridization probes and the appropriate vectors are known in the art. Hybridization probes may be labelled by a variety of well known reporter groups.

Such diagnostic tools may be used to detect any disorder associated with hyperproliferation such as cancer, a reproductive disorder or a disorder associated the bone, or may be used in the prevention and/or treatment of immune pathologies. In a particular aspect, the polynucleotides encoding NuSAP may be useful in assays that detect activation or induction of various cancers. Such assays may also used to evaluate the efficacy of a particular therapeutic treatment of the diseases enumerated herein above. In order to provide a basis for the diagnosis of disease associated with expression of NuSAP, a normal or standard profile for expression in the respective tissues is established. Once disease is established and a treatment protocol is initiated, assays may be repeated on a regular basis to evaluate progression. The results obtained may be used to show the efficacy or inefficacy of the treatment over a period ranging from several days to months.

With respect to cancer, the presence of a relatively high amount of transcript of NuSAP in a biopsied tissue from an individual may indicate a predisposition of said individual for the development of the disease, or may provide a means for detecting the disease prior to the appearance of actual symptoms. A more definitive diagnosis of this type may allow health professionals to employ preventive measures or aggressive treatment earlier thereby preventing the development of further progression of the cancer.

Additional diagnostic uses for oligonucleotides designed from the sequences encoding NuSAP involve the use of PCR. Such oligomers may be chemically synthesized, generated enzymatically, or produced from a recombinant source. Oligomers will preferably consist of two nucleotide sequences, one with sense and another with antisense orientation, employed under optimized conditions for identification of a specific gene or condition, as well known to those skilled in the art.

Methods which may also be used to quantify the expression of NuSAP include radiolabeling, fluorescent or immunolabeling of nucleotides, coamplification of a control polynucleotide, and standard curves on which the experimental results are interpolated, such as disclosed for instance by Melby et al. in J. Immunol. Methods (1993) 159:235-244 and Duplaa et al. in Anal. Biochem. (1993) 229-236. Also, the polynucleotide sequences which encode NuSAP may be used to generate hybridization probes which are useful fro mapping the naturally occurring genomic sequence. The sequences may be mapped to a particular chromosome or to a specific region of the chromosome using well known techniques such as, but not restricted to fluorescence in situ hybridization (hereinafter referred as FISH) or FACS.

The following examples are only for the purpose of describing particular embodiments of this invention and are not intended to limit its scope, which is only defined by the appending claims. This invention is not limited to the particular methodologies, protocols, cell lines, vectors, and reagents described in the following examples as these may vary according to the general common knowledge of the skilled person. Most of the technologies used herein-after are described in Current Protocols in Molecular Biology (1999), John Wiley & Sons, New York.

### EXAMPLES

Materials and methods used were as follows:

Cell Culture and Treatments

All culture reagents, unless otherwise mentioned, were obtained from GIBCO BRL (Life Technologies, Grand Island, N.Y.). Murine osteoblast-like MC3T3-E1 cells (Sudo et al. (1983). J. Cell Biol. 96:191-198) were purchased from Riken Cell Bank (H. Kodama, Japan), and maintained in αMEM supplemented with 10% heat-inactivated FCS and penicillin-streptomycin (100 U/ml and 100 mg/ml, respectively). Sub-confluent MC3T3-E1 cell cultures are defined to be in the proliferation state, while confluent and mineralising cells have entered the differentiation stage. COS-1 and NIH3T3 cells (American Type Culture Collection, Manassas, Va.) were cultured respectively in Dulbecco's modified eagle medium (hereinafter DME) and DME-F12, plus 10% heat-inactivated FCS. For NIH3T3 cells, the medium was supplemented with glutamax (2 mM) and glucose (4.5 g/l). Cultures were grown at 37° C. in a humidified atmosphere containing 5% CO₂ in air.

Synchronized MC3T3-E1 cells were obtained by a double thymidine block (Stein, et al. (1990) in “Cell Growth and Division, a practical approach”. R. Baserga, editor. IRL Press at Oxford University Press, Oxford. 133-137; Ryoo et al. (1997) J. Cell. Biochem. 64:106-116). Cells were exposed twice to 2 mM thymidine (Calbiochem-Novabiochem Corp., La Jolla, Calif.) and were harvested at varying time points after release from the growth arrest.

Growth arrest of MC3T3-E1 cells by serum starvation was accomplished by changing complete medium (10% FCS) to medium containing 0.1% FCS in subconfluent cultures. The quiescent cells were released from growth arrest by adding complete medium (10% FCS), whereafter cells were harvested at the indicated time points. Growth arrest was also induced by treating subconfluent MC3T3-E1 cultures with 1 mM hydroxyurea (Calbiochem-Novabiochem Corp).

To obtain mitotic COS-1 cell populations, 1 mM hydroxyurea was added to the culture for 18 h, whereafter cells were washed and released in growth medium for 7.5 h. Nocodazole was obtained from Calbiochem-Novabiochem Corp., taxol from Sigma Chemicals Co. (St Louis, Mo.), and aphidicolin from Roche Molecular Biochemicals (Mannheim, Germany).

1α,25(OH)₂D₃ is commercially available from Hoffman-La Roche Inc., Nutley, N.J.).

Differential Display and RACE

Differential display was performed essentially as previously described (Liang et al. (1993) Nucleic Acids Res. 21:3269-3275). Briefly, total cellular RNA was extracted from cell cultures using TriZol LS (GIBCO BRL) and was treated with RNase free DNase I (Roche Molecular Biochemicals) to remove any residual DNA contamination. After phenol/chloroform extraction and ethanol precipitation, RNA was dissolved in H₂O and was reverse-transcribed using Superscript II (GIBCO BRL) with the following anchor primer set: 5′-T₁₂MG-3′ where M represents A, C or G. The resultant cDNAs were amplified by PCR in the presence of [³³P]dATP (Amersham Pharmacia Biotech., Uppsala, Sweden) using 5′-T₁₂MG-3′ and the following arbitrary decamer: 5′-ACGGTCATAG-3′. PCR products were separated on a 6% polyacrylamide/urea sequencing gel; the gel was dried and the products were visualized by autoradiography. Bands exhibiting differential expression were excised from the dried gel, and DNA in the gel slice was eluted and reamplified using the same set of primers. The PCR products were ligated into the cloning vector pGEMTEasy (Promega Corp., Madison, Wis.). Screening of positive clones was carried out as previously described (Shoham et al. (1996) Biotechniques 20:182-184). Amplitaq (Perkin-Elmer Biosystems, Foster City, Calif.) was used in all PCR steps.

The 3′-primers, that were used in the 5′-RACE kit (GIBCO BRL) were designed based on the 3′-untranslated region (UTR) sequence obtained from the differential display fragment. NuSAP mRNA was reverse-transcribed using the following primer: 5′-CAGATGGCAAATACATG-3′. PCR amplification of the resultant cDNA was performed with the following gene specific 3′-primer: 5′-CAGAACAAGGCTCTTATAAGC-3′. The PCR product was ligated into the cloning vector pGEMTEasy.

DNA Sequence Analysis

Plasmid DNA was purified using a Nucleobond Plasmid Midikit (CLONTECH Labs, Palo Alto, Calif.), and the sequence of all cloned fragments was determined via the dideoxynucleotide chain termination method using a fluorescent cycle sequencing kit and the ALF Express DNA sequencer from Amersham Pharmacia Biotech. The nucleic acid and deduced protein sequence were compared with known sequences in the databanks using the BLAST algorithms (Altschul (1997) Nucleic Acids Res. 25:3389-3402). The GCG software package (Genetics Computer Group, Inc., Madison, Wis.) was used for all sequence assembly and analysis.

Northern Analysis

Total cellular RNA was extracted from cell cultures using TriZol LS. Foetal mice and soft tissues from adult mice (C57BL/6; 8 wk old) were directly homogenized in TriZol (GIBCO BRL), while femurs and tibias were ground to a fine powder prior to homogenization, followed by total cellular RNA extraction. 18 μg of denatured RNA was applied per lane, separated by electrophoresis in 1% agarose/formaldehyde gels and transferred to positively charged nylon membranes (ICN Pharmaceuticals Inc., Costa Mesa, Calif.) as previously described (Ausubel (1999) Current Protocols in Molecular Biology, John Wiley & Sons, New York. 4.9.1-4.9.13). Membranes were prehybridized and then hybridized for 18 hours at 42° C. with cDNA probes, labeled with [³²P] dCTP by random priming (Amersham Pharmacia Biotech.), in 50% formamide-containing solutions. The membranes were then washed and subjected to autoradiography at −70° C. To assess equal loading, blots were rehybridized with a radiolabelled glyceraldehyde 3-phosphate dehydrogenase (GAPDH), β-actin or an 18 S ribosomal probe. The following cDNA probes were used: human histone H4, pFO002 (Pauli et al. (1989) J. Cell Physiol. 139:320-328) mouse osteocalcin, p923 (Celeste et al. (1986) EMBO J. 5:1885-1890) and a 0.8 kb NuSAP BglII/AspI restriction fragment.

In Vitro Transcription and Translation

The coding region of NuSAP was amplified using pyrococcus furiosus (hereinafter referred as Pfu) polymerase (Stratagene, La Jolla, Calif.) with the following specific primers, 5′-CGAGATTGCAGAACGCGATGAC-3′ and 5′-TTAGAGTAAAGGAACAGACAGGG-3′. The PCR fragment was blunt-end ligated into the pCRScript plasmid (Stratagene) and sequenced to verify NuSAP sequence and proper reading frame. The plasmid DNA was transcribed and translated using the TNT T7 reticulocyte lysate system (Promega Corp.) with translation grade L-[³⁵S]cysteine (1000 Ci/mmol; Amersham Pharmacia Biotech). Translation products were separated by SDS-polyacrylamide gel electrophoresis and processed for fluorography. Luciferase DNA was used as a positive control. Treatment with calf intestine alkaline phosphatase (GIBCO BRL) was for 30 minutes at 37° C.

Construction of NuSAP Vectors

The NuSAP sense expression vector was designed to contain the coding sequences of NuSAP fused upstream to the Myc- and His-tag of the pcDNA3.1(−)/Myc-His B plasmid (Invitrogen Corp., Carlsbad, Calif.). The following specific primers incorporating a 5′-NotI and a 3′-BamHI site were used in the amplification reaction: 5′-TAAGGATCCAAGTTCACACCCAGGTTTCTTCG-3′ and 5′-ATAAGAATGCGGCCGCCATGGCCGTCCCCTCTGCAGAGGAGCTG-3′. The digested PCR fragment was inserted into the complementary sites of the pcDNA3.1(−)/Myc-His B plasmid.

The NuSAP antisense vector was engineered by cloning the NotI-BamHI insert from the pCRScript vector used in the transcription and translation experiments (see supra) into the corresponding sites of the pcDNA3.1(−)/Myc-His A plasmid. The NuSAP-GFP fusion vector was designed to contain the coding sequences of NuSAP fused upstream to the green fluorescent protein (GFP) sequence of the pEGFP-N1 plasmid (CLONTECH). The following specific primers incorporating a 5′-SacI and a 3′-BamHI site were used in the PCR reaction: 5′-TAAGGATCCAAGTTCACACCCAGGTTTCTTCG-3′, and 5′-CTTGAGCTCTACCCGAGATTGCAGAACGCG-3′. The digested PCR fragment was inserted into the complementary sites of the pEGFP-N1 plasmid.

The myc- and flag-tagged NuSAP vectors were constructed after removing the GFP-tag by digesting with BamHI and NotI, and inserting a myc and flag adapter duplex containing these restriction sites. The following oligonucleotides were used: 5′-GATCTAGAACAAAAACTCATCTCAGAAGAGGATCTGTGA-3′ and 5′-GGCCTCACAGATCCTCTTCTGAGATGAGTTTTTGTTCTA-3′ for the myc-tagged vector and 5′-GATCTAGACTACAAGGACGACGACGACAAGTGA-3′ and 5′-GGCCTCACTTGTCGTCGTCGTCCTTGTAGTCTA-3′ for the flag-tagged vector.

In all cases, Pfu polymerase (Stratagene) was used in the amplification reaction. All plasmids were sequenced to verify junctions and to ensure the proper NuSAP sequence.

HuNuSAP cDNA containing the protein coding sequence was cloned via PCR using cDNA derived from RNA isolated from bone tissue of an adult human female. Cloned in pcDNA3.1(−)Myc-His using the following primers:

1) 5′-CGCGGATCCTTCAGCCAAAATGAGGCCCCTTC-3′

2) 5′-ATAAGAATGCGGCCGCCATGGTCATCCCCTCTCTAGAGGAGC-3′

[³H]-Thymidine Incorporation

COS-1 and NIH3T3 cells were plated in flat bottomed 96 well plates (Nalge Nunc International, Rochester, N.Y.), respectively at 2000 and 750 cells per well. After one day, cells were transfected using Fugene 6 (Roche Molecular Biochemicals) according to the manufacturer's instructions with the NuSAP-sense and -antisense expression vectors and pcDNA3.1(−)/Myc-His B plasmid as a control. For each condition there were at least 6 replicates and experiments were repeated at least 3 times. Plasmid DNA derived from independent DNA preparations was also used.

[³ H]-thymidine incorporation was performed 48 hours post transfection. 1 μCi [³H]-thymidine (2 Ci/mmol; ICN Pharmaceuticals Inc.) was added to each well for 4 hours at 37° C. in 5% CO₂, whereafter the cells were harvested using a micro cell harvester (Filtermate Universal Harvester, Packard Instrument Co., Meriden, Conn.) on glass filter membranes (Unifilter, Packard Instrument Co.). After drying and addition of Microscint-O scintillation liquid (Packard Instrument Co.), microplates were counted in a TopCount Microplate Scintillation Counter (Packard Instrument Co.).

Antibodies

The following antibodies were used to detect epitope tagged NuSAP: for immunoprecipitation analysis, a rabbit polyclonal Ab (pAb) to GFP (Molecular Probes, Eugene, Oreg.) was used and for immunofluorescence analysis, a rabbit pAb against c-myc (Santa Cruz Biotechnology, Santa Cruz, Calif.), and a mouse mAb to flag (M2) (Sigma Chemicals Co.) were used. Goat pAb against lamin A/C were obtained from Santa Cruz Inc. (Santa Cruz, Calif.). The mouse monoclonal anti-α-tubulin antibody (Blose (1984) J. Cell Biol. 98:847-858) was purchased from Amersham Pharmacia Biotech. The mouse monoclonal anti-C23 (nucleolin) antibody was purchased from Santa Cruz Biotechnology, Inc. The secondary Texas Red-labeled goat anti-mouse antibody was purchased from Molecular Probes, Inc. BSA (fraction V) was purchased from Sigma Chemicals Co. The mouse monoclonal anti-His (C-terminal) antibody was purchased from Invitrogen Corporation. A 5-Bromo-2′-deoxy-uridine (BrdU) labeling and detection kit (Roche Molecular Biochemicals) was used according to the manufacturer's instructions to detect BrdU incorporated into cellular DNA. Actin was detected using Texas Red-X-Phalloidin (Molecular Probes).

Immune Fluorescence Microscopy

COS-1 cells were plated in Lab-Tek II chamber slides (Nalge Nunc International) and transfected with the NuSAP-GFP fusion vector using Fugene 6 according to the manufacturer's instructions. Immunofluorescence analysis was performed 24 hours after transfection. Cells were briefly washed in phosphate buffered saline (PBS), fixed in methanol/acetone (1/1) and were air dried. Cells were then washed 3 times in 0.1% Tween 20/PBS, and incubated for 20 minutes at room temperature with 0.5% bovine serum albumin (BSA)/PBS, pH 7.4. Thereafter cells were incubated with primary antibody at 2 μg/ml in 0.5% BSA/PBS for 1 hour at room temperature, washed 4 times in 0.5% BSA/PBS, and incubated with the secondary antibody at 6 μg/ml in 0.5% BSA/PBS for 1 hour at room temperature. Cells were then washed 4 times in PBS prior to post-fixation in 3.7% paraformaldehyde/PBS for 30 minutes. The chromosomes were stained by incubating the cells for 5 minutes in PBS containing 2 μg/ml Hoechst 33258 (Sigma Chemicals Co.). Cells were mounted in DAKO fluorescent mounting medium (DAKO, Glostrup, Denmark).

Alternatively, cells were fixed and permeabilized in 0.4% glutaraldehyde/0.5% Triton X100/PBS for 15 minutes at room temperature. After incubation in 0.5 mg NaBH4/ml PBS (10 minutes), and blocking in 5% BSA/PBS (10 minutes), cells were incubated with primary antibodies for 1 h. Cells were washed and after incubation with 50% normal serum (Dako) (10 minutes), cells were incubated with secondary antibodies conjugated to Alexa-488 or -546 dye (Molecular Probes) (1 hour) and finally mounted using Dako mounting medium. Antibodies and normal serum were diluted in 0.5% Tween20/PBS. Analysis was done on a Nikon inverted microscope Diaphot 300 (Plan Apo 60/1.40 oil) connected to a Bio-Rad MRC1024 confocal microscope and images were captured by LaserSharp (version 3.2) and processed using Adobe Photoshop 5.5 (Adobe Systems Inc., San Jose Calif.). The images showing a-tubulin localization were taken using a Leitz DM microscope and were recorded using a cooled charge-coupled device camera (Photometrics Inc., Tucson, Ariz.) and IP-Lab software. The presence of the filamentous network was also confirmed using other fixation and permeabilization agents which included 0.8% paraformaldehyde/0.5% 7 Triton X100/PBS, methanol/acetone 1:1 (volume/voume), and 4% paraformaldehyde. For BrdUrd labeling, cells were incubated with 10 μmol BrdUrd (15 minutes) prior to fixation, and treated with 0.07N NaOH (2.5 min) prior to the BSA block.

Nuclear Extraction for Microscopy

24 hours after transfection, COS-1 cells were briefly washed in PBS and subjected to permeabilization and cell extraction as described (Fey et al. (1984) J. Cell Biol. 98:1973-1984). Cells were extracted with CSK buffer (0.5% Triton X-100 in 10 mM Pipes, pH 7.0, 100 mM NaCl, 300 mM sucrose, 3 mM MgCl₂, and 1 mM EGTA (Ethylene glycol-bis(2-aminoethyl)-N,N,N′,N′-tetraacetic acid) containing 1 mM phenylmethylsulfonyl fluoride (PMSF) twice for 15 minutes each at 4° C. Thereafter, RNA or DNA were digested twice with 100 μg/ml RNase A and 100 μg/ml DNase I (Roche Molecular Biochemicals) respectively for 30 minutes each in digestion buffer (CSK buffer with 50 mM NaCl) at 30° C. Digested chromatin was extracted at 4° C. with 250 mM ammonium sulfate in the digestion buffer for 10 minutes and again for 5 minutes. Cells were fixed for immunofluorescence microscopy as described above.

Immunoprecipitation and Western Blot Analysis.

Cell extracts were prepared 24 hours after transfecting COS-1 cells with the NuSAP-GFP expression vector or the empty GFP vector, as a control. After washing in ice-cold PBS, cells were harvested by scraping in PBS. The cells were pelleted and resuspended in ice-cold lysis buffer (50 mM Tris HCl (pH 7.4), 0.5% Triton X100, 0.1% NP-40, 150 mM NaCl, 1 mM EDTA, 0.25% gelatine, 1 mM PMSF, 10 μg/ml aprotinin, 10 μg/ml leupeptin, and 1 μg/ml antipain). Cells were homogenized by passing the mixture several times through a G26-needle and subsequently centrifuged at 10000× g (15 minutes at 4° C.). Extracts were then precleared (40 minutes at 4° C.) using control serum and protein-A or -G agarose beads (Santa Cruz), and subsequently incubated with specific antibodies and beads (1 hour at 40° C.) under rotation. Beads were pelleted, washed three times in lysis buffer and the precipitated protein complexes were eluted by boiling in SDS sample buffer. Proteins were seperated by sodium dodecyl sulfate polyacrylamide gel electrophoresis (SDS-PAGE), and were transferred to a nitrocellulose membrane. Membranes were probed with the relevant primary and secondary antibodies (Dako) and developed by enhanced chemiluminescence (ECL system, New England Nuclear) according to the manufacturers instructions.

Fluorescence-Activated Cell Sorter (FACS) Analysis

Sub-confluent COS1 cells were collected by trypsinization 24 or 48 hours after transfection with the NuSAP-GFP or empty GFP expression vectors. Cells were first fixed for 45 minutes at 4° C. in CellFIX solution (Becton Dickinson) for the preservation of GFP fluorescence. Subsequently, cells were fixed for 2 hours at 4° C. in 70% ethanol as required for DNA staining. After washing twice in PBS, cells were resuspended in a solution containing 25 μg/8 ml propidium iodide (Calbiochem), 100 μg/ml RNase A (Sigma) and 0.5% Triton X100 and incubated for 45 minutes at room temperature. Cells were immediately analyzed for DNA content by flow cytometry (FACScan, Becton Dickinson). Analysis of DNA content was restricted to GFP-expressing cells, and was repeated at least three times using independently transfected cell populations. Different plasmid DNA preparations were also tested.

In Situ Hybridization

Digoxigenin-11-UTP-labelled single-strand RNA probes were prepared using a DIG RNA labeling kit (Roche Molecular Biochemicals) according to the manufacturer's instructions.

The NuSAP cDNA fragment was used to generate antisense and sense probes, which were reduced to an average size of 100 bases by limited alkaline hydrolysis.

Embryo's and organs of adult mice were fixed in 2% paraformaldehyde in PBS by perfusion, incubated at 4° C. overnight and then washed, dehydrated and embedded in paraffin wax (Hoang et al. (1998) Dev. Dyn. 212:364-372). Sections of 8 μm were cut (Leica RM 2155, Leica Microsystems Inc., Deerfield, Ill.), transferred to positively charged Optiplus slides (Biogenex, Sam Ramon, Calif.) and dried overnight at 37° C. After deparaffinization, sections were treated with 10 μg/ml of proteinase K (Roche Molecular Biochemicals) for 10 minutes at room temperature and thereafter fixed in 4% paraformaldehyde in PBS for 10 minutes. Next, the slides were washed with PBS and treated with acetic anhydride (0.25%). Hybridization was performed at 70° C. in 50% formamide, and washes were carried out at a stringency of 0.2× SSC containing 50% formamide at 70° C. A DIG nucleic acid detection kit (Roche Molecular Biochemicals) was used according to the manufacturer's instructions to detect signals.

### Example 1

### Identification and Isolation of the Mouse nusap cDNA

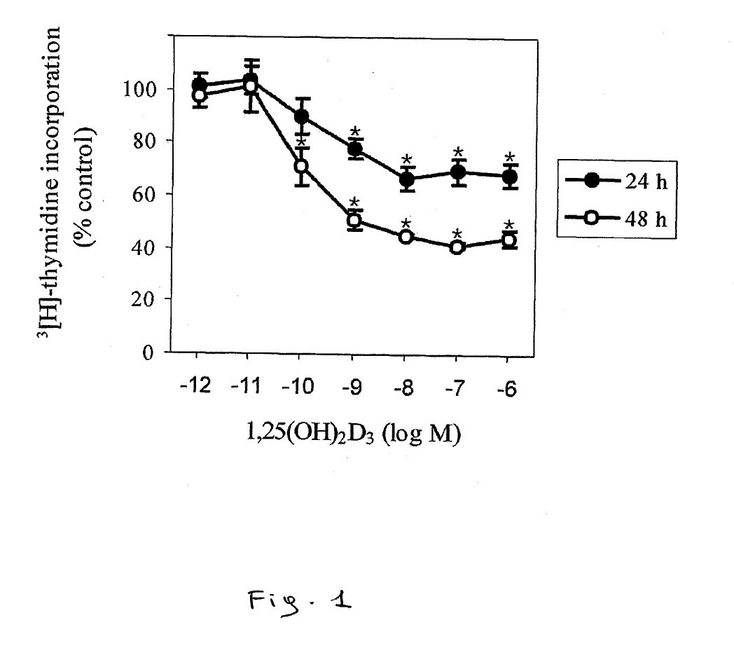

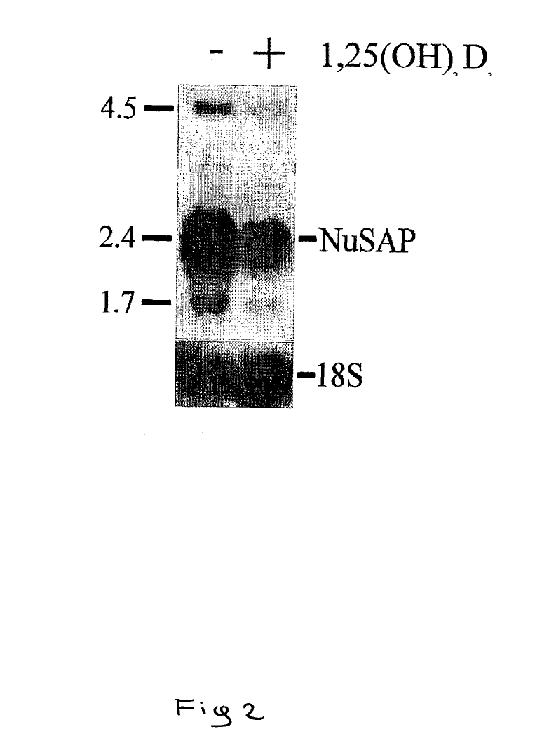

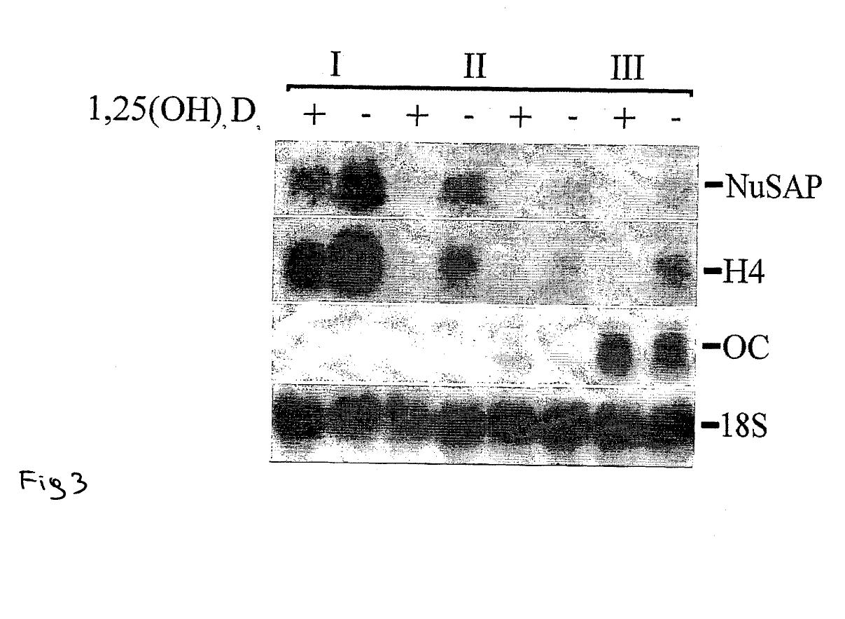

Treatment of mouse osteoblastic MC3T3-E1 cells with 1α,25(OH)₂D₃ inhibited the proliferation of these cells as measured by [³H]-thymidine incorporation (FIG. 1). Significant effects were already observed after 24 hours and this inhibition became more pronounced with increasing time. To obtain more insight in this anti-proliferative effect, we used mRNA differential display analysis to compare gene expression after treatment with 1α,25(OH)₂D₃ (10⁻⁸ M) or vehicle for 24 hours. A cDNA fragment that showed reduced levels after 1α,25(OH)2D₃ treatment was cloned. Similarly, the gene level of this particular cDNA clone is also lower in a differential display of undifferentiated cells (subconfluent cell culture) compared with differentiated cells (confluent, mineralized cell culture). Northern analysis using this cDNA fragment as a probe confirmed the inhibition of nusap gene expression after treatment and also revealed the presence of three transcripts: one major 2.4 kb transcript and two minor transcripts of 1.7 kb and 4.5 kb (FIG. 2). Comparing nusap gene expression with molecular markers for growth (histone H4) and differentiation (osteocalcin). in long term osteoblast cultures (reviewed in Stein, et al. (1996) Physiol. Rev. 76:593-629), showed a clear association of nusap gene expression with proliferation (FIG. 3).

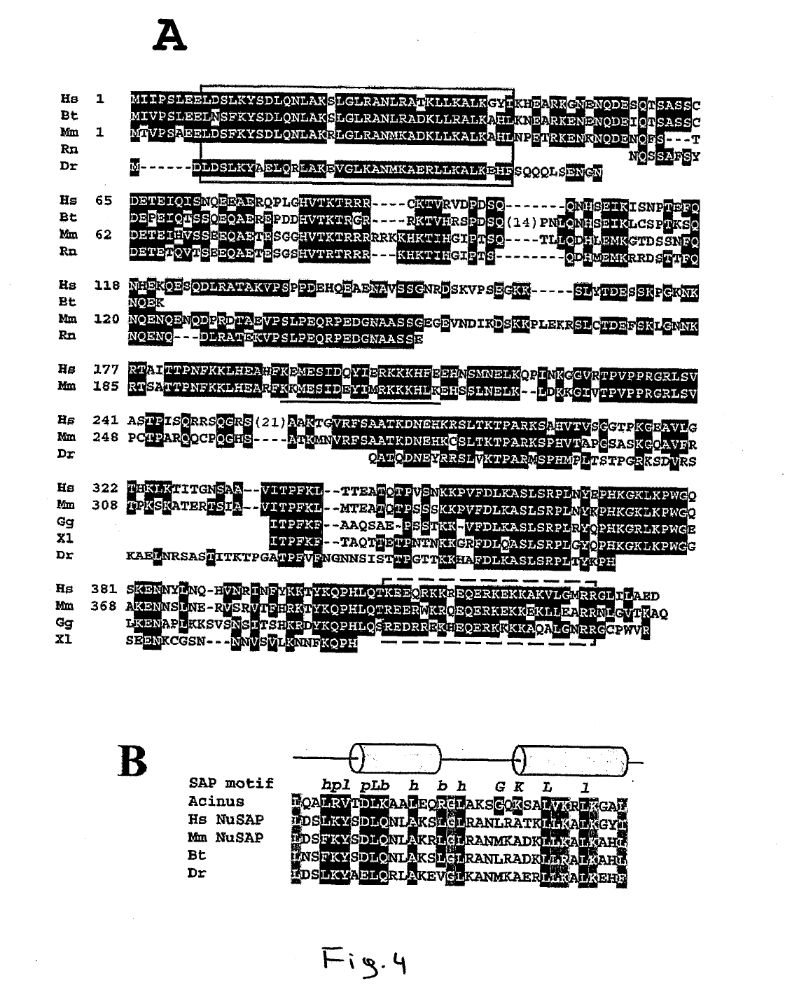

The full-length sequence of the major 2.4 kb transcript was obtained via 5′RACE using gene specific primers based on the differential display cDNA fragment. The entire cDNA was sequenced and the single long open reading frame was analyzed (FIG. 4). Both mouse and human sequences were cloned and BLASTP (Altschul et al. (1997) Nucleic Acids Res. 25:3389-3402) searches revealed that the predicted protein sequences were identical to the sequences of recently submitted, but unpublished proteins present in lymphocytes (GENBANK accession no. AAG31285 & AAG25874).

### Example 2

### Analysis of the Mouse NuSAP Protein Sequence

Translation of nusap cDNA is predicted to start at the first methionine positioned at nucleotide 71 and to terminate at nucleotide 1352. The first ATG lacks an ideal Kozak context (Kozak. (1991) J. Biol. Chem. 266:19867-19870) but an accessible 5′UTR (49% GC) can however compensate for the less favorable ribosome binding-site context. The 70 bp 5′UTR is followed by a 1281 bp coding region and a 785 bp 3′UTR containing two consensus polyadenylation signals (AATAAA) positioned at nucleotides 1885 and 2123 (SEQ ID NO:1). Differential usage of these signals can clarify the minor 1.7 kb transcript (FIG. 2). The presence of in-frame termination codons in both 5′- and 3′-UTRs supports the correct assignment of the initiator methionine.

The major open reading frame (ORF) contained in the nusap cDNA predicts a protein of 427 amino acids with a calculated molecular weight of 48.6 kD and a predicted isoelectric point (pI) of 9.9. The highly charged protein has 141 charged residues, of which 85 are basic. The many lysine residues (K), which make up about 12% of the protein, are more abundant towards the C terminus. The protein possesses few major hydrophobic amino acid residues. A hydropathy plot, according to Kyte et al. in J. Mol. Biol. (1982) 157:105-132, did not show any apparent membrane-spanning region nor was a N-terminal signal sequence detected.

Examination of the predicted sequence also identified two types of potential nuclear localization signals (NLSs) (Dingwall et al. (1991) Trends Biochem. Sci. 16:478-481): a single stretch of four basic residues that appears several times and one bipartite nuclear localization signal (NLS) (FIG. 4).

The search for protein motifs also indicates the presence of a sequence in the aminoterminal part of NuSAP, which is highly reminescent of a SAP motif (FIG. 4). The SAP motif is a DNA binding motif involved in chromosomal organization (Aravind et al., cited supra). The putative SAP motif of NuSAP contains many spaced leucine residues which comply with the consensus sequence. It has a similar predicted secondary structure (via PredictProtein) where two alpha-helices are formed separated by a turn, and resides at the N-terminus of the protein, a feature that is observed in many other proteins containing this motif (FIG. 4). The ‘invariant’ glycine residue of the SAP motif, which is found between the two helices, is not conserved and is replaced by a hydrophobic residue (leucine or methionine) probably resulting in a variant turn.

The NuSAP protein sequence contains multiple consensus protein phosphorylation sites. Several putative casein kinase II, protein kinase C (PKC) and one cAMP- and cGMP-dependent protein kinase phosphorylation sites were identified in NuSAP using the PROSITE algorithm (Hofmann et al. in Nucleic Acids Res. (1999) 27:215-9). Based on studies of cdc2 kinase phosphorylation sites in the major nucleolar protein nucleolin, a consensus sequence for mitotically active cdc2 kinase (X-T/S-P-X-K/R) was developed (Peter et al. (1990) Cell. 60:791-801). The NuSAP sequence possesses three of these consensus cdc2 kinase phosphorylation sites, two of which are completely conserved between the mouse and human sequence. The PROSITE databank search also identified several potential N-myristoylation, one N-glycosylation site and one putative glycosaminoglycan attachment site.

The protein seems to be evolutionary conserved as there is significant sequence similarity (>60% at the protein level) between the mouse NuSAP protein and the human homologue

### Example 3

### Characterization of the Mouse nusap Gene Product

To confirm that the presumptive open reading frame can produce a protein of the expected molecular mass of 48.6 kD, in vitro transcription and translation was performed using nusap cDNA containing the predicted ORF. A protein of approximately 55 kD was detected on SDS PAGE. The difference observed between the apparent and predicted molecular mass can be due to the multiple consensus phosphorylation sites present in the protein sequence. To test this hypothesis we treated the in vitro transcription and translation product with alkaline phosphatase for 30 minutes and observed a small band shift, indicating a change in molecular mass by the removal of phosphate groups. The difference between predicted and apparent molecular mass can in part be ascribed to this post-translational modification. Another plausible explanation is that, due to the highly charged nature of the protein, it exhibits an apparent molecular mass on SDS PAGE significantly higher than predicted from its amino acid sequence as has been described for other proteins (Mortillaro et al. J. Biol. Chem. 273:8183-8192). Alternatively, other post-translational modifications can account for the difference in molecular mass.

### Example 4

### Proliferation- and Cell Cycle-Specific Expression of the Mouse nusap Gene in MC3T3-E1 Cells

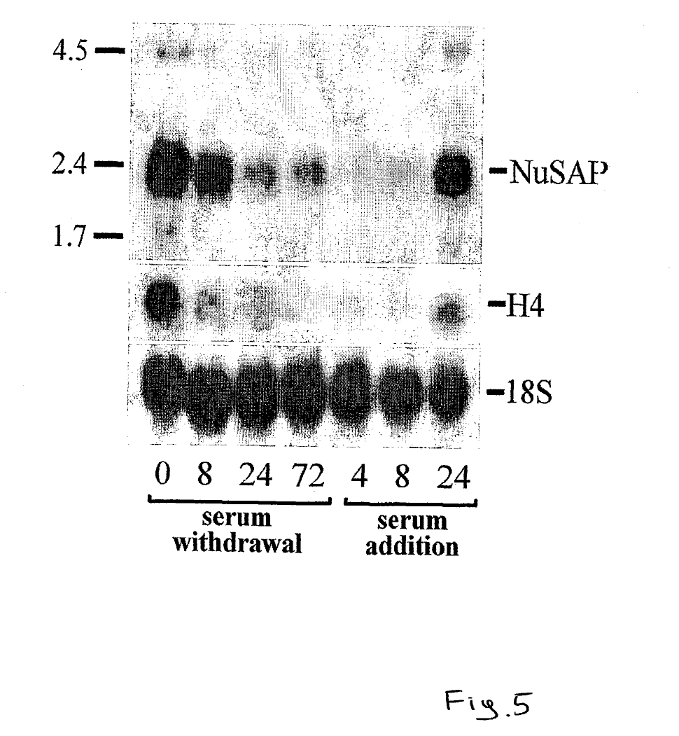

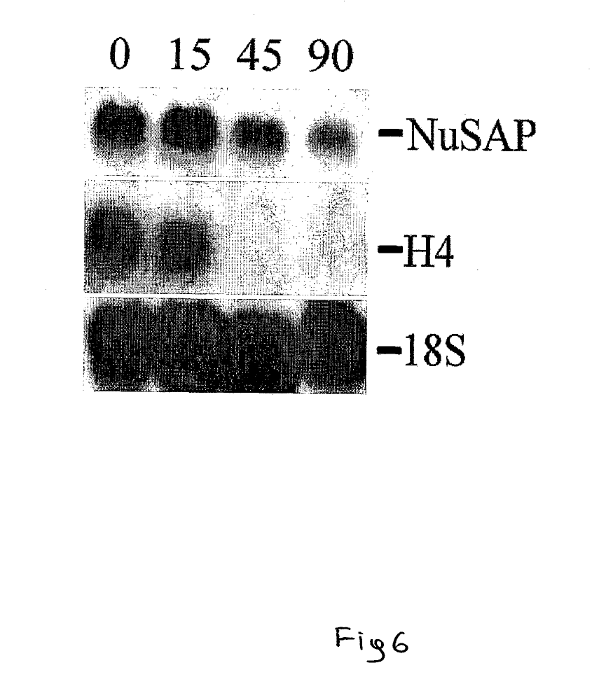

The expression of the nusap gene was shown to correlate with the proliferation phase during osteoblast development (FIG. 3). To further characterize this proliferation-specific expression of nusap growth arrest was induced in MC3T3-E1 cells by serum starvation and RNA was extracted at the indicated time points (FIG. 5). A clear reduction in nusap RNA levels was observed 8 hours after withdrawal of serum. When complete growth medium was added to the quiescent MC3T3-E1 cells, allowing them to proliferate, near maximal levels of nusap gene expression were reached within 24 hours. A comparable expression pattern was observed for the histone H4 gene. When growth arrest of cells was induced by hydroxyurea, a ribonucleotide reductase inhibitor (Timson, (1975) Mutat. Res. 32:115-132), both nusap and histone H4 RNA levels quickly declined (FIG. 6). Within 45 minutes of treatment histone H4 RNA levels became undetectable, and nusap gene expression was markedly down-regulated. Knowing that histone H4 gene expression is directly coupled to DNA replication these data suggest that the expression of nusap is linked to DNA replication, but not directly coupled to it.

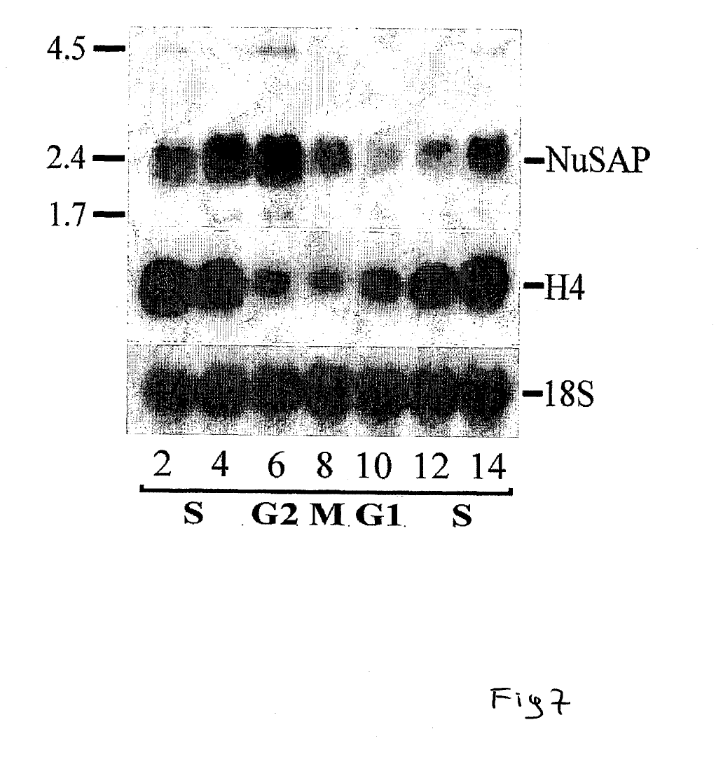

Based on the association with cell proliferation, we further examined whether nusap RNA levels were altered during progression of the cell cycle. A double thymidine block was used to synchronize MC3T3-E1 cells at the G1/S phase boundary (FIG. 7). The different cell cycle phases were assigned by determining the message levels of histone H4, a S phase marker, as has been described by Ryoo et al. in (1997) J. Cell. Biochem. 64:106-116) using the same cell line. Northern analysis showed high RNA levels of nusap 4 to 6 hours after release from growth arrest. Comparing nusap RNA levels with those of histone H4 revealed that nusap gene expression was up-regulated during the S and G2 phase of the cell cycle (FIG. 7). After release for 8 to 10 hours (M/G1 phases) nusap message levels declined, to increase again in the subsequent S phase. These data indicate that nusap gene expression is regulated during the cell cycle.

### Example 5

### Mouse nusap Expression is Tissue-Specific

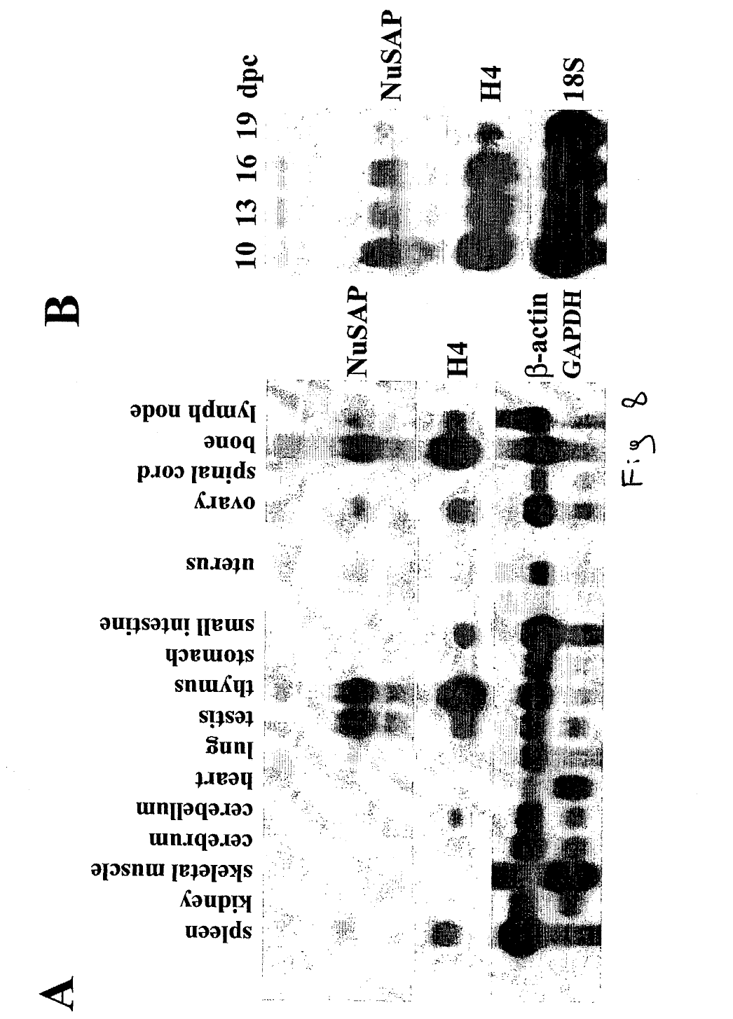

To further characterize the nusap gene and its biological activity, we examined the expression pattern of nusap in adult mice tissues. Northern analysis of total (FIG. 8) and poly(A)+ RNA extracted from adult mice tissues (C57BL/6 strain) showed that the gene was expressed in organs of the immune system (thymus, spleen, lymph node), organs of the reproductive system (testis, ovary, uterus), lung, small intestine, and bone. The highest levels were detected in the thymus, testis and bone. Interestingly, despite a high expression in the testis, the 4.5 kb minor transcript was not detectable. Histone H4 gene expression was also analyzed as a marker of the proliferation state of the organs (FIG. 8). Comparing the levels of expression of the two genes, it is evidenced that in some organs, like the thymus, they are well correlated. There is however no strict correlation in every organ examined: the central nervous system and kidney show higher RNA levels for histone H4 than for nusap, while in the testis, the opposite is observed.

Northern analysis of total RNA extracted from different developmental stages of whole mouse embryo's (C57BL/6) showed that the nusap gene was expressed at higher levels in younger embryos (FIG. 8). The level of expression of the nusap gene declined with increasing days post-coitum (dpc): 10 dpc>13 dpc>16 dpc>19 dpc. The histone H4 gene showed a comparable expression pattern (FIG. 8b). Analysis of expressed sequence tag (EST) entries at GenBank using the BLASTN algorithm (Altschul et al. (1997) Nucleic Acids Res. 25:3389-3402.) revealed that nusap is expressed as early as the 2-cell stadium in mice (accession No. AI505293).

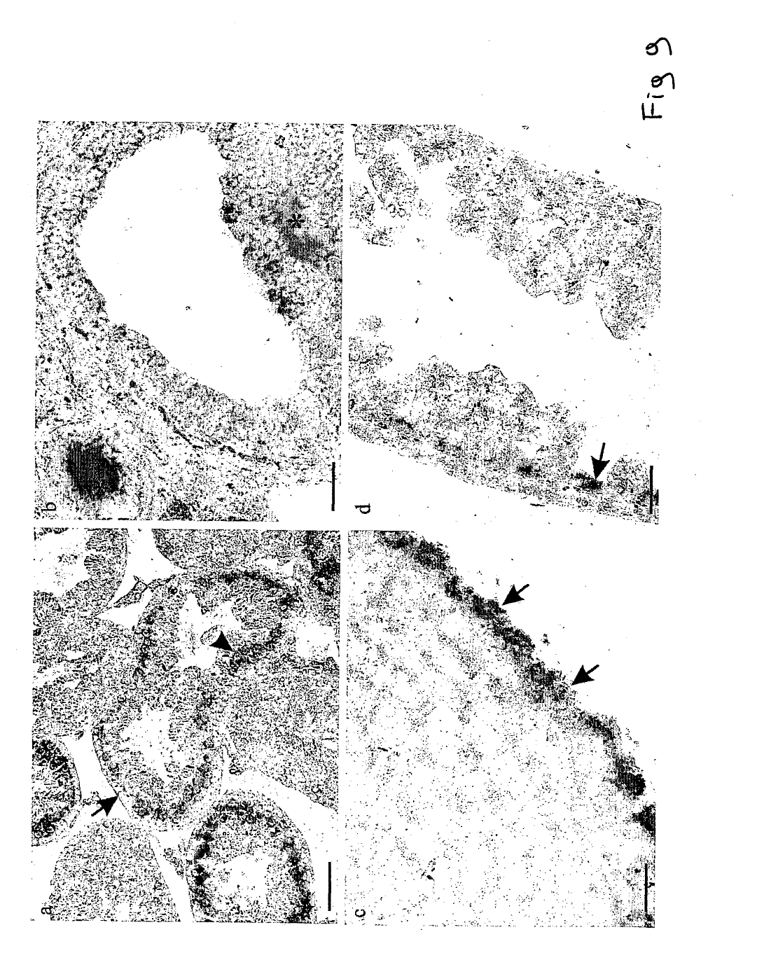

To investigate whether nusap gene expression was cell-specific, we analyzed tissues from embryo's and adult mice (C57BL/6) by in situ hybridization (FIG. 9). Interestingly, in the adult testis (FIG. 9a) the mitotically active spermatogonia within the seminiferous tubule showed little or no expression of the gene. High expression was confined to the primary spermatocytes undergoing meiosis. By staging the seminiferous tubule, high expression of the nusap gene is predicted to correlate with the late pachytene stage of prophase during meiosis. The absence of expression of nusap in some tubules can be explained by the fact that not all tubules undergo the same stage of development. Little or no expression was observed in the interstitial cells of the seminiferous tubules, in the Sertoli cells, in the seminiferous epithelium, and in cells in spermiogenesis.

In tissue sections of adult ovary there was little or no nusap expression in the proliferating follicular epithelial cells and in the granulosa cells. High expression was confined to the oocytes (FIG. 9b). The thecal cells surrounding the follicular epithelial cells were also negative for nusap expression.

In the adult mouse thymus (FIG. 9c), high expression of nusap was localized to the sub-capsular outer cortex region containing actively proliferating immature T-stem cells. Little or no nusap-positive staining was observed in the cortex, where cells are probably in a resting stage, and in the medulla, where most cells are differentiated.

In the small intestine of a 19 dpc mouse embryo (FIG. 9d), the absorptive epithelial cells of the villi showed little or no staining in contrast to the mitotically active cells in the crypts that showed strong staining for nusap expression. The cells of the lamina propria, the supporting layer underlying the intestinal epithelium showed little or no nusap expression. In all tissue sections examined, no staining was observed when the sense probe was used.

These data show that nusap gene expression is tissue-specific and restricted in reproductive organs to cells in meiosis, and in other tissues to mitotically active cells.

### Example 6

### NuSAP is Overexpressed in Different Types of Cancer

A 200 bp fragment of human nusap cDNA was labeled with [32P] labeled random primers. This probe was used to hybridize a panel of cDNA's derived from normal and cancer tissues according to the manufacturers instructions (matched tumor/normal expression array, (Clontech, Palo Alto). Quatitative detection was performed on a phosphorimager.

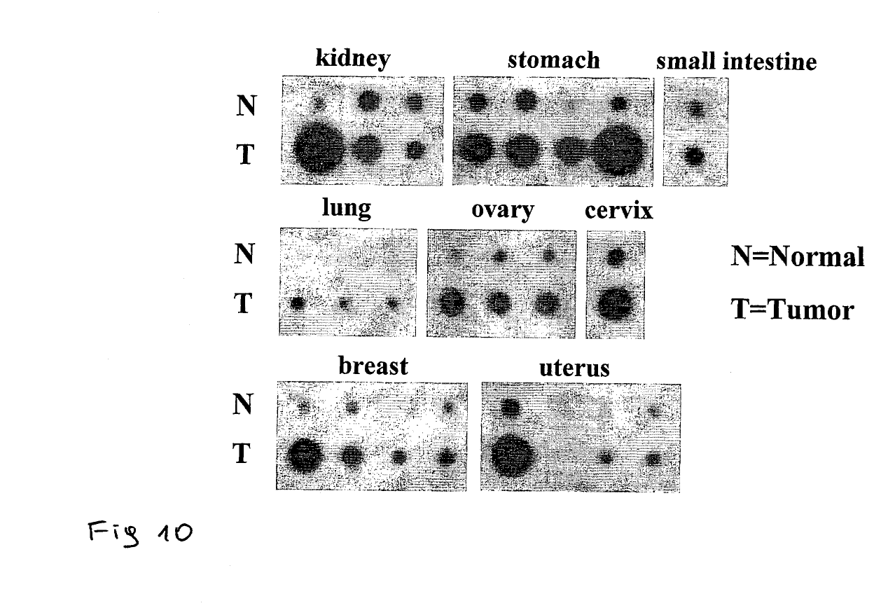

nusap was hybridised to a membrane on which cDNA, synithesised from 68 tumors and coresponding normal tissue from the same individual, have been immobilised (Clontech matched tumor/normal expression array). This blot contains cDNA pools of tumors from kidney, breast, prostate, uterus, ovary, cervix, colon, lung, stomach, rectum, and small intestine. As shown in FIG. 10, in 8 of the 11 tissues represented in this blot, a clear overexpression of nusap is seen in tumors compared to normal tissue. As can be seen from this figure, the ratio of nusap expression in tumors compared to normal tissue differs from tumor to tumor. this property can be used to diagnose and discriminate different types of cancer in one organ.

FIG. 10 shows the elevated expression levels of nusap in cDNA pools of human tumor tissue (designated as T) as compared to the expression of nusap incDNA pools obtained from the same tissue from healthy individuals (designated as N). Quantitative detecture of nusap is detected by exposure on a phosphorimager.

### Example 7

### NuSAP is a Nuclear Protein with a Discrete Distribution

Immunofluorescence microscopy was used to analyze the subcellular distribution of mouse NuSAP in MC3T3E1 and COS1 cells. An expression vector producing full length mouse NuSAP fused at its C-terminus to a GFP-tag was constructed and used in transient transfection experiments. Most of the observations were confirmed using myc- and flag-tagged NuSAP expression vectors.

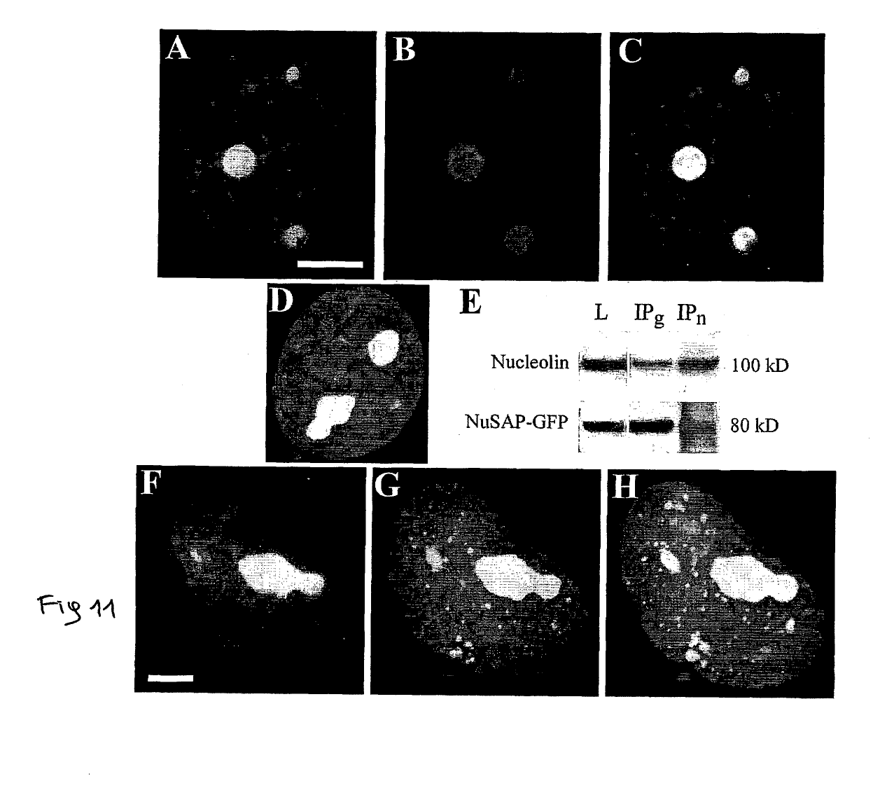

As expected from the presence of a potential nuclear localization signal, fluorescence from GFP-tagged NuSAP was restricted to the nucleus (FIGS. 11A and D), as evidenced by immunostaining for lamin A/C (FIG. 11D). Within the nucleus, GFP-tagged NuSAP was predominantly observed within the nucleolus as demonstrated by nucleolin colocalization (FIGS. 11A-C). Compared to nucleolin, that especially resides at the fibrillar component (Ginisty et al. (1999) J. Cell Sci. 112 761-772), GFP-tagged NuSAP was found to localize both to the fibrillar component and the fibrillar center.

To investigate whether NuSAP and nucleolin interacted directly with each other, co-immunoprecipitation experiments were carried out on extracts from transfected COS1 cells (FIG. 11E). Immunoprecipitation with antibodies directed to the GFP-tag not only resulted in the precipitation of the NuSAP-GFP fusion protein (80 kDa) but also clearly demonstrated the presence of nucleolin (100 kDa). Reversely, when antibodies directed to nucleolin were used, the immunoprecipitate contained not only nucleolin but also NuSAP-GFP fusion protein.

Detailed confocal laser scanning microscopy revealed that fluorescence from GFP- (and myc- or flag-) tagged NuSAP, as opposed to nucleolin immunostaining, was in addition observed in organized structures within the nucleoplasm of transfected interphase COS1 cells. Successive horizontal plane sections of the nucleus taken at 1 μm (FIGS. 11F-H) z axis intervals showed the presence of a filamentous network that was most clearly visible towards the periphery of the nucleus (FIGS. 4F and G-01). The filaments were seen crisscrossing the nucleus, though in sections taken at the center of the nucleus (FIG. 11H) a more diffuse fluorescence pattern was observed suggesting a possible further ramification of the network at the nuclear core. The filaments were distinct from the nuclear lamins as demonstrated by immunostaining for lamins A/C and B. Also, no colocalization was observed with tubulin and actin using specific anti-tubulin antibodies and phalloidin (data not shown). These staining patterns were observed in every transfected interphase cell using different fixatives and permeabilizing agents.

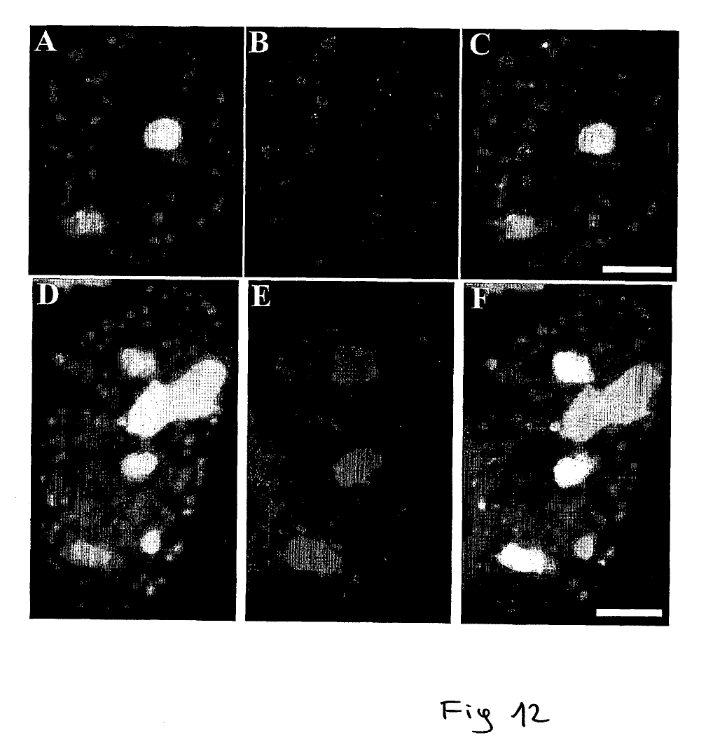

In addition to its nucleolar and filamentous localization, GFP-tagged NuSAP was also observed in multiple small foci throughout the nucleoplasm. Confocal analysis of staining for NuSAP and incorporated BrdUrd, a marker for newly replicated DNA, showed that BrdUrd foci localized adjacent to NuSAP foci (FIGS. 12A-C). Treatment of transfected cells with aphidicolin, a DNA synthesis inhibitor, (20 μg/ml for several hours) resulted in loss of immunostaining for BrdUrd though fluorescence from GFP-tagged NuSAP persisted (data not shown) indicating an indirect role of the protein in DNA replication. As a likely consequence of overexpression, the small NuSAP positive foci sometimes clustered together with BrdUrd foci in very large aggregates (FIG. 12D), which at reduced illumination intensities were clearly granular.

The observations described above were similarly made using myc- and flag-tagged NuSAP. Interestingly, GFP-tagged NuSAP shows a specific intracellular localization, not only during interphase, but also during mitosis. Double staining for GEP-tagged NuSAP and a-tubulin, in cells fixed during mitosis, showed that during metaphase and anaphase, NuSAP localizes to the microtubules of the spindle apparatus and the spindle poles. NuSAP's name derives from this initial observation: NUclear Spindle-Associating Protein.

### Example 8

### NuSAP Relocalizes to the Chromosomes and Microtubules During Mitosis

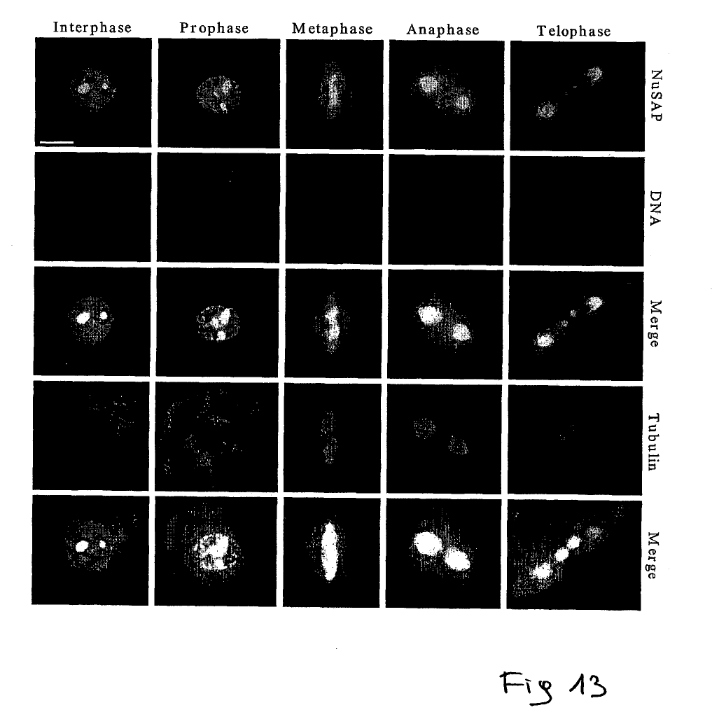

In a next step the subcellular distribution of GFP-tagged NuSAP during mitosis was investigated. During this phase of the cell cycle certain nucleolar proteins (i.e. B23 and fibrillarin) localize to the chromosome periphery, while others localize to the nucleolar organizing region (Hernandez-Verdun et al. (1994) Bioessays. 16:179-185). Synchronization of transfected COS-1 cells with hydroxyurea, confirmed that during interphase the protein is predominantly localized to the nucleolus. In addition these experiments showed that as cells enter mitosis NuSAP translocates and associates with the mitotic chromosomes (FIG. 13). During early prophase, chromatin condensation begins at the nuclear envelope (Comings et al. (1971) Exp. Cell Res. 63:471-473) and continues until the individual chromosomes are visible. During this period GFP-tagged NuSAP associates with the chromosomes and fluorescence is most intense. In metaphase, the chromosomal association disappears and NuSAP localizes to the spindle associating with the microtubules and the brightly fluorescing spindle poles (FIG. 13). This was shown via colocalization experiments with α-tubulin, a microtubule subunit (Blose et al. (1984) J. Cell Biol. 98:847-858.). This spatial organization continues through anaphase, but during telophase NuSAP, in addition to being associated with the microtubules, which is especially visible in the midbody, also relocalizes to the chromosomes.

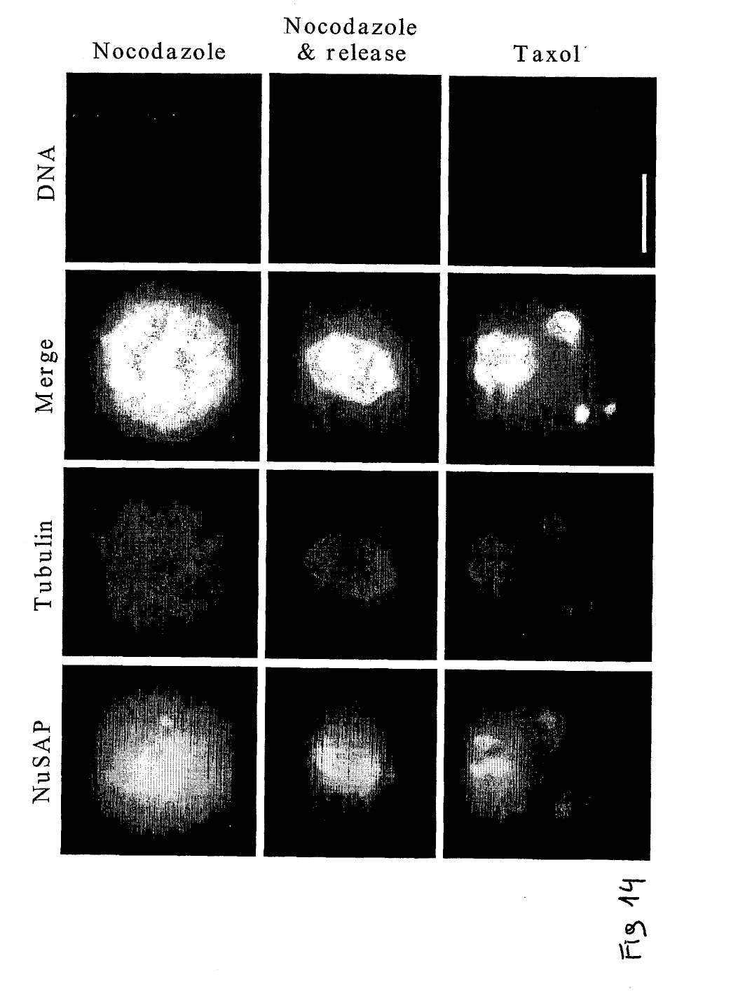

Further evidence for an association of NuSAP with the microtubules of the mitotic spindle was obtained using both nocodazole, a microtubule depolymerizer (Jordan et al. (1992) J. Cell Sci. 102:401-416), and taxol, a microtubule stabilizer (De Brabander et al. (1981) Proc. Natl. Acad. Sci. USA. 78:5608-5612). When COS-1 cells transfected with GFP-tagged NuSAP were synchronized to obtain a population in mitosis and thereafter incubated with nocodazole (10 μM) for 1 hour NuSAP and α-tubulin redistributed throughout the cell (FIG. 14). However, when nocodazole was washed out and the mitotic spindles were allowed to re-assemble for 1 h NuSAP relocated to the microtubules and spindle poles. Furthermore, incubation of mitotic cells with taxol (5 μM for 1 hour) which stabilizes microtubules and induces mitotic asters which are devoid of centrosomes (De Brabander et al., cited supra) resulted in NuSAPs translocation to these structures (FIG. 14). Neither nocodazole nor taxol treatment affected the distribution of NuSAP in the nucleoli during interphase.

### Example 9

### NuSAP-Sense and -Antisense Expression Alters DNA Synthesis

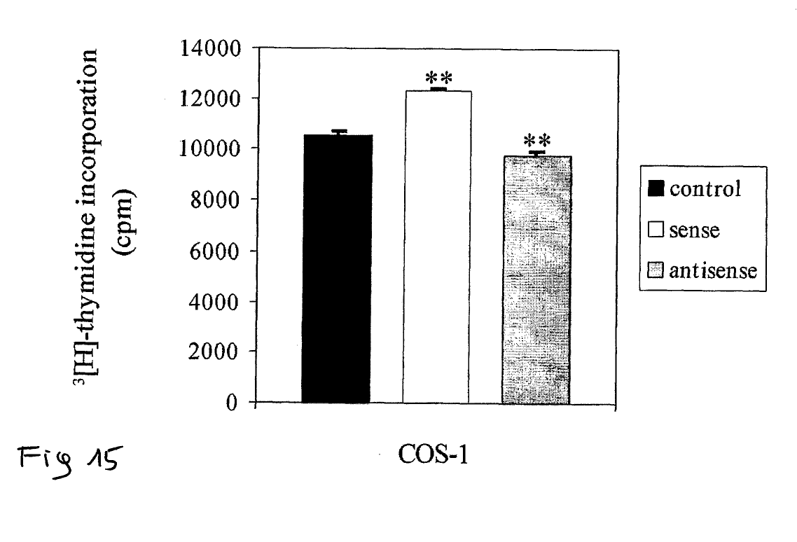

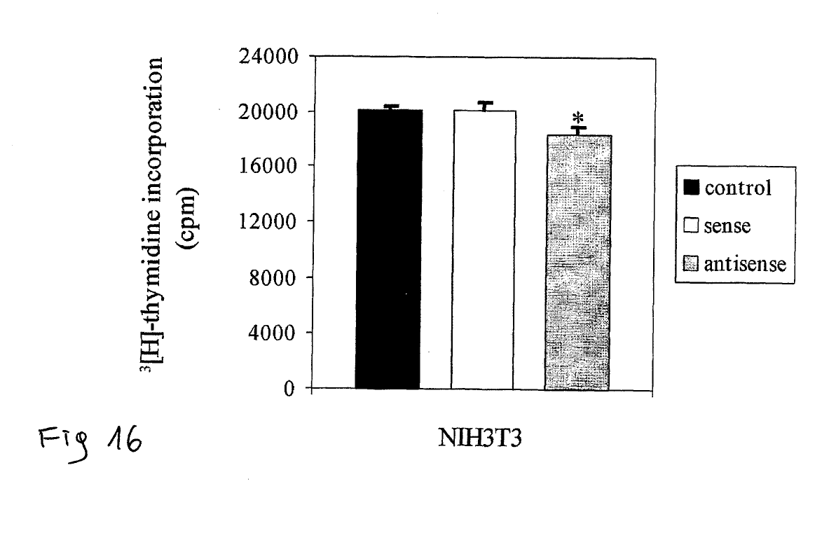

To evaluate the role of NuSAP in cell proliferation and DNA replication, COS-1 and NIH3T3 cells were transfected with nusap-sense, -antisense, and control (without an insert) expression vectors and tested for [³H]-thymidine incorporation 48 hours later (FIGS. 15 and 16). COS-1 cells transfected with the nusap sense expression vector showed an increase of 14% in thymidine uptake (P<0.01) compared to the control. Expression of antisense nusap in both COS-1 and NIH3T3 cells inhibited the thymidine uptake by 7.7% (P<0.01) and 8.4% (P<0.05) respectively, compared to the control. These data show that NuSAP has a moderate positive effect on cell proliferation.

### Example 10

### Effect of NuSAP Overexpression on the Cell Cycle

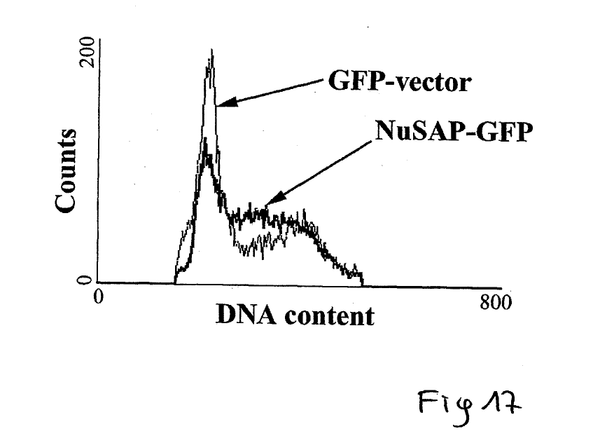

Because fluorescence from GFP-tagged NuSAP is found juxtaposed to sites of BrdUrd incorporation in transfected COS1 cells, the question we asked was whether NuSAP overexpression affected cell cycle progression. Using FACS analysis, COS1 cells, transfected with the NuSAP-GFP expression vector or the empty GFP-expression vector (as a control), were analyzed both at 24 and 48 hours post transfection. Cells expressing the NuSAP-GFP fusion protein showed a clear increase in the S-phase population, relative to the control, that was most obvious at 48 hours post transfection (FIG. 17). Arrest in S-phase progression was accompanied by a decrease in the population of cells in the G0/G1-phase.

### Example 11

### Generation of a NuSAP Mouse Knockout

The NuSAP targeting vector contains a 5′ flanking 5.2-kb SacI-Eco restriction enzyme (RI) and a 3′ flanking 4.5-kb EcoRI fragment encompassing exon 2 (containing the ATG). A ‘floxed’ cassette containing the neomycin phosphotransferase gene will be inserted at the EcoRI site upstream of exon 2 and a third lox site will be introduced downstream of exon 2 at another EcoRI site that will be inactivated. This vector will be μg/ml) and gancyclovir (2 μM) and correct targeting was be assessed by Southern analysis using an external 1.5-kb SacI probe and an internal 1-kb NcoI-PstI probe. Targeted ES cell clones were electroporated with a Cre-recombinase expression cassette and Southern analysis demonstrated correct excision of the ‘floxed’ neo cassette. Chimeric mice were generated by morula aggregation and bred for germline transmission. NuSAP deficient mice were obtained by crossing NuSAP^{lax} mice with PGK-cre mice (Lallemand, Y., Luria, V., Haffner-Krausz, R. and Lonai, P. (1998) Transgenic Res 7:105-112.), while tissue-specific inactivation of NuSAP was obtained by crossing NuSAP^{lox} mice with transgenic mice expressing cre tissue-specific.

### Example 12

### Generation of a Transgenic Mouse Over-Expressing NuSAP

This example provides a transgenic rodent, in particular a mouse, whose genome contains a NuSAP gene coding for SEQ ID NO:1 or its rodent homologue for the expression of the protein according to SEQ ID NO:2 or its rodent homologue, further comprising a promoter in front of the gene for modulating the expression of the protein in comparison with wild type.

The NuSAP targeting vector contains a 5′ flanking 5.2-kb SacI-Eco restriction enzyme (RI) and a 3′ flanking 4.5-kb EcoRI fragment encompassing exon 2 (containing the ATG) in which an strong inducible promoter (e.g. tetracycline promoter) is placed in front of the NuSAP gene. This vector is electroporated in R1 embryonic stem cells and selection was performed with G418 (200 μg/ml) and gancyclovir (2 μM) and correct targeting was assessed. Overexpression of nusap in these cells was tested prior to aggregation. Chimeric mice are generated by morula aggregation and bred for germline transmission.

Expression of nusap in these mice occurs after addition of tetracycline and can be used as a model for screening anti cancer drug compounds since a mouse overexpressing nusap is more susceptible to be affected by carcinogens.

The teachings of the various above examples may be summarized as follows. The present invention identifies a novel nuclear gene named nusap with the following characteristics: (i) the expression is tissue-specific (especially in organs of the immune and reproductive system, and in bone) (ii) expression is associated with high RNA levels in meiotic and actively proliferating cells where (iii) its expression is cell cycle-dependent and (iv) where NuSAP protein associates with the nucleolus and elaborate fibrous network in nucleus (v) altering hereby cell proliferation in a still undefined way. NuSAPs remarkable associations with different subcellular structures indicate that it is a highly dynamic protein: during the interphase of proliferating cells, NuSAP localizes predominantly to the nucleolus but is also found adjacent to sites of DNA replication; in mitotic cells, NuSAP relocates initially to the condensing chromosomes and then associates with the microtubules of the mitotic spindle to eventually reassociate with the chromosomes at the end of mitosis.

NuSAP Has a Putative SAP Motif

Especially interesting in the light of NuSAP's proposed structural role in coordinating spatial regulation of DNA replication, is the potential existence of a SAP motif within the protein. This is a putative DNA binding motif involved in chromosomal organization (Aravind et al., cited supra). Assignment of the motif is based on sequence similarity, predicted secondary structure similarity, and a N-terminal localization. The presence of this motif points to a participation in additional nuclear processes, besides DNA replication. NuSAP's gene expression pattern relates to this aspect. A structural role in DNA recombination is envisaged based on its tissue-specific expression in lymphoid tissues where immunoglobulin and T cell receptor gene rearrangements occur (Jeggo et al. in Bioessays (1995) 17:949-957), and in reproductive tissues where meiotic recombination take place. Consistent with such a role, is the increased gene expression observed in the G2-phase of the cell cycle, where mitotic recombination events during replication-coupled-repair occur (Hendrickson Am. J. Hum. Genet. (1997) 61:795-800).

Involvement in DNA Replication

The role of NuSAP in DNA replication is supported by its localization adjacent to sites of BrdU incorporation. Replication factors that have-been shown to localize to these sites include proliferating cell nuclear antigen (PCNA) (Bravo et al. in (J. Cell Biol. (1987)105:1549-54) and DNA ligase I (Montecucco et al. in EMBO J. (1995) 14:5379-5386). Interestingly, the cell cycle regulators cyclin A and Cdk 2 have also been shown to localize to these sites and they provide a direct link between cell cycle regulation and DNA replication (Cardoso et al. in Cell. (1993) 74:979-992). Localization of NuSAP adjacent to sites of BrdU incorporation is however partial: NuSAP foci are more abundant than replication foci, which indicates that not all NuSAP is participating in the replication process. NuSAPs involvement in DNA replication is also supported by the following observations. First, cell synchronization experiments reveal that nusap expression is upregulated during the replicative phase of the cell cycle, as determined by examining histone H4 gene expression, an S-phase specific marker (Stein et al. in Curr. Opin. Cell Biol. (1992) 4:166-173). Second, nusap gene expression shows a strong correlation with the degree of cell proliferation (or DNA replication). In serum starvation experiments, nusap RNA levels declined when growth factors were withdrawn, and increased upon readdition. Similarly hydroxyurea treated cells showed a rapid decline in RNA levels of both nusap and histone H4. Third, the tissue-specific expression often shows a correlation with histone H4 expression, a molecular marker whose expression is linked to DNA replication. In addition, the in situ data reveal that the cell-specific expression correlates with proliferative cells. In the thymus, expression is restricted to the immature T-stem cells which are actively dividing and in the embryonic small intestine, high NuSAP expression is restricted to the actively dividing cells in the crypts. Finally, sense and antisense studies in cultured cells show respectively an increase (COS-1) and decrease (NIH3T3 and COS-1) of [³H]-thymidine incorporation into cellular DNA, a reflection of DNA replication.

With respect to the exact function of NuSAP within the complex process of DNA replication, some cells that were in S-phase, as monitored by BrdU incorporation, showed that NuSAP was not localized to these intra-nuclear foci, indicating that this association is restricted to a certain period of the S-phase. In addition, participation in other fundamental cellular events like transcription and splicing is not excluded. These processes also occur within distinct domains in the nucleus (Berezney et al. in Int. Rev. Cytol. (1995) 162A:1-65) and the NuSAP foci that do not co-localize to sites of DNA replication may participate in these processes.

Vitamin D Actions Linked to DNA Replication

The possible role of NuSAP in promoting DNA replication also provides a new mechanism underlying the anti-proliferative action of 1α,25(OH)₂D₃. To date these effects of 1α,25(OH)₂D₃ have been assigned to its induction of several cyclin-dependant kinase inhibitors, including p21 and p27 (Liu et al. in Genes Dev. (1996) 10:142-153.; Segaert et al. in J. Invest. Dermatol. (1997) 109:46-54), either directly or indirectly, and to its alteration of cyclin levels (Wang et al. in Cancer Res. (1996) 56:264-247; Verlinden Mol. Cell. Endocrinol. (1998) 142:57-65), ultimately resulting in an arrest of the cell cycle in the G1 phase. The present data show that 1α,25(OH)₂D₃ significantly down-regulates nusap expression 24 hours after treatment when DNA replication declines.

Possible Function in DNA Recombination

The data obtained, however also show that NuSAP has additional roles besides DNA replication. From the tissue-specific expression pattern of nusap it is evident that its expression not always correlates with histone H4 expression. The central nervous system and the kidney show higher RNA levels for histone H4 than for NuSAP, while in the testis the opposite is observed. In addition, the cell-specific expression pattern in the testis and ovary further points to a role for NuSAP in the meiotic process. In the adult testis, high expression is confined to the primary spermatocytes in the late pachytene stage of meiosis I. During pachytene, the homologous chromosomes which are in synapsis remain closely associated until recombination is completed (Moens Bioessays. (1994) 16:101-106). The examples show that NuSAP is involved in the recombination event. Further support for this role is the high expression of NuSAP in the thymus, spleen and lymph node, where the gene product is considered to be involved in lymphoid cell-specific reactions such as recombination during immunoglobulin and T cell receptor gene rearrangements (Jeggo Bioessays. (1995) 17:949-957) and/or immunoglobulin class switching (Harriman et al. in Annu. Rev. Immunol. (1993) 11:361-384). Furthermore, the high expression of nusap observed during the G2-phase of the cell cycle implies a role in DNA repair coupled to DNA replication: during the late S- and the G2-phase, the ploidy of the cell increases to 4n, allowing homologous recombination to proceed of an undamaged template (Hendrickson Am. J. Hum. Genet. (1997) 61:795-800). Interestingly, some nucleolar proteins have been linked to cellular recombination events: B23 (nucleophosmin) and nucleolin (C23) as part of the SWAP complex in B-cell switch recombination (Blose et al. in J. Cell Biol. (1984) 98:847-858), SC65 in meiotic recombination (Ochs et al. in Mol. Biol. Cell. (1996) 7:1015-1024), and Sir2 and Pch2 in yeast rDNA and meiotic recombination (San-Segundo et al. in Cell. (1999) 97:313-324).

Recently, proteins with functions in both replication and recombination have been identified (Beernink et al. in Trends Biochem. Sci. (1999) 24:385-389; Kodadek, Trends Biochem. Sci. (1998) 23:79-83), although studies have generally been focused on prokaryotes and lower eukaryotes. A plausible working hypothesis is that these proteins bind to single stranded DNA (ssDNA) facilitating hereby the assembly of enzyme complexes (for DNA replication and recombination) to the ssDNA (Beernink et al., cited supra).

Recruitment to the Nucleolus

During the interphase of proliferating cells, NuSAP localizes predominantly to the nucleolus. This was confirmed through colocalization studies with nucleolin, a major nucleolar protein. The nucleolus has long been believed to be a site solely dedicated to rDNA transcription and ribosome biogenesis (reviewed in Shaw et al. (1995) Annu. Rev. Cell Dev. Biol. 11:93-121). Recently however, additional nucleolar functions have been recognized (Pederson (1998) J. Cell Biol. 143:279-281) especially in yeast where mitotic and meiotic roles for the nucleolus in cell cycle control and transcriptional regulation have been defined (reviewed in Garcia et al. (1999) Cell. 97:825-828). The emerging view of these studies is that the nucleolus serves as a privileged site for both the recruitment and exclusion of regulatory complexes. NuSAP can in a similar way be recruited to the nucleolus and released when required in processes like DNA replication. NuSAP's interaction with nucleolin has been demonstrated. Its close association with the nucleolar remnant within the nuclear matrix, and its lack of association with extractable DNA and RNA within the nucleolus, further supports this. Such a recruitment will make a rapid response possible, independent of the transcriptional and translational regulatory circuits. The multiple consensus phosphorylation sites within NuSAP indicates that such a response can be triggered by phosphorylations. Interestingly, a role in the initiation of DNA replication has been assigned to nucleolin, based on its ability to form a complex with Replication Protein A after cell stress (Daniely et al. (2000) J. Cell Biol. 149, 799-809).

Association wit the Nuclear Matrix

The protein was named NuSAP based on the initial observation of being a nuclear spindle-associated protein. The strong conservation of protein sequence in vertebrates indicates an essential function in higher eukaryotes. A structural role within the nucleus is proposed, based on the localization of the mouse protein to an unique intranuclear filamentous network. Its organization within the nucleus is reminiscent of the structural framework observed in nuclear matrix preparations (He et al. (1990) J. Cell Biol. 110:569-580; Nickerson et al. (1995) Int. Rev. Cytol. 162A:67-123; Wan et al. (1999) Proc. Natl. Acad. Sci. USA. 96:933-938). The nuclear matrix is the non-chromatin nuclear structure that likely serves as a scaffold, determining the general organization of the nucleus. By positioning and transporting nuclear factors to appropriate spatial domains within the nucleus, the nuclear matrix is likely to contribute to the efficient assembly of functional complexes. The molecular composition of the structural components of the nuclear matrix has not yet been clearly determined, but candidates do exist. The filament-forming Tpr protein is one such candidate. It is a nuclear pore complex associated protein that is organized into filaments extending 100-350 nm into the nucleus (Cordes et al. (1993) J. Cell Biol. 123:1333-1344; Zimowska et al. (1997) J. Cell Sci. 110, 927-944; Strambio-de-Castillia et al. (1999) J. Cell Biol. 144:839-855). It has also been shown to have a function in nuclear trafficking of macromolecules (Strambio-de-Castillia et al., cited supra; Bangs et al. (1998) J. Cell Biol. 143:1801-1812). A second group of candidates composing the nuclear matrix are the nuclear lamins. They not only form a filamentous structure under the nuclear envelope, but also exist as foci within the nuclear interior. However, the presence of an extensive internal lamin network has yet to be demonstrated. Our observations of the distribution pattern of NuSAP indicate that it is clearly distinct from the filament-forming proteins mentioned above. As expected, the filaments also proved to be distinct to actin filaments and microtubules. At the core of the nucleus NuSAP's localization to filaments is less evident, but some filaments remain discernible. Electron microscopic analysis will bring clarification to this issue. Previous studies have suggested that the nuclear matrix helps coordinate the spatial organization of DNA replication (Berezney et al. (1981) Exp. Cell Res. 132:1-13; Vaughn et al. (1990) Nucleic Acids Res. 18:1965-1969). NuSAP's localization to multiple small foci embedded within the network, which are seen adjacent to sites of newly replicated DNA, suggest a role for the protein in the replication process

Association with the Mitotic Spindle and Chromosomes

With respect to the functional significance of NuSAPs association with different cellular structures (condensing chromosomes during prophase, mitotic spindle during metaphase and anaphase, and the chromosomes and midbody during telophase) during mitosis the multiple associations form a pathway through which daughter cells are ensured equal amounts of NuSAP. Alternatively, association to the respective cellular structures is of functional importance and would imply a critical role for NuSAP in determining progression of the cell cycle. At the start of mitosis, nucleolar NuSAP translocates to the condensing chromosomes. It has been shown that histone H1, with a possible chromosome condensing activity (Bradbury et al. (1974) Nature. 247:257-261), becomes phosphorylated by Cdc 2 kinase as cells enter mitosis (Langan et al. (1989) Mol. Cell. Biol. 9:3860-3868). NuSAP contains three consensus Cdc2 phosphorylation sites that were characterized by Peter et al. (1990) Cell. 60:791-801 based on Cdc2 phosphorylation of nucleolin during mitosis. The existence of consensus Cdc 2 phosphorylation sites in NuSAP may be linked to its association with condensing chromosomes during mitosis. During metaphase and anaphase, the association of NuSAP with the mitotic spindle and its poles is particularly evident. This association with microtubules is sensitive to treatment with nocodazole, a microtubule depolymerizer (Jordan et al. (1992) J. Cell Sci. 102:401-416) and is induced by treatment of mitotic cells with taxol, a microtubule stabilizer capable of inducing microtubule asters (De Brabander et al. in Proc. Natl. Acad. Sci. USA. 78:5608-5612).

Taken together, we show that NuSAP is a novel nuclear protein that has a role in DNA replication and most likely in other cellular processes including recombination. Based on the highly basic nature of the protein, we assign to NuSAP a structural role in these processes. The multiple consensus phosphorylation sites make its regulation during these processes possible.

1. An isolated and purified eukaryotic polynucleotide encoding a Nuclear Spindle-Associating Protein, the said polynucleotide containing the nucleic acid sequence shown in SEQ ID NO:1 or the nucleic acid sequence shown in SEQ ID NO:3 or an allelic variant thereof.

2. A Nuclear Spindle-Associating Protein having the amino acid sequence SEQ ID NO:2 or the amino acid sequence SEQ ID NO:4 or coded by an allelic variant of a polynucleotide containing the nucleic acid sequence shown in SEQ ID NO:1 or the nucleic acid sequence shown in SEQ ID NO:3.

3. A eukaryotic polynucleotide according to claim 1, further comprising a polynucleotide which codes for at least a portion of a protein or a marker, the combination of both polynucleotides being arranged in such a way that a fusion protein results after expression.

4. A vector comprising at least a eukaryotic polynucleotide according to claim 1.

5. A fusion protein resulting after expression of the eukaryotic polynucleotide according to claim 3.

6. Use of a eukaryotic polynucleotide according to claim 1, a homologue, a portion or a mutation thereof, or a protein according to claim 2, a homologue, a variant, a portion or a mutation thereof, or a fusion protein according to claim 5 for the manufacture of a diagnostic tool for the detection of a cancer of a tissue selected from the group consisting of kidney, stomach, small intestine, lung, ovary, cervix, breast and uterus.

7. Use of a eukaryotic polynucleotide according to claim 1 or a protein according to claim 2, or a fusion protein according to claim 5 for the manufacture of a diagnostic tool.

8. A pharmaceutical composition comprising a eukaryotic polynucleotide according to claim 1 and a pharmaceutically acceptable carrier.

9. A process for the production of a protein according to claim 2, comprising culturing a host cell containing an expression vector comprising a eukaryotic polynucleotide according to claim 1, and obtaining said protein from the cell or the culture medium.

10. A host cell, other than a human germ line cell, being transformed with a eukaryotic polynucleotide according to claim 1.

11. A method of controlling gene expression of a eukaryotic polynucleotide according to claim 1, comprising the step of administering to a mammal a pharmaceutical composition comprising at least a therapeutic amount of a vitamin D.

12. A method of influencing the biological activity of a Nuclear Spindle-Associating Protein according to claim 2 or a fusion protein according to claim 5, comprising the step of administering to a mammal a pharmaceutical composition comprising at least a therapeutic amount of a vitamin D.

13. A method according to claim 11 or claim 12, wherein the said method is for modulating the process of DNA replication in the said mammal.

14. A method according to claim 11 or claim 12, wherein the said method is for modulating the process of DNA recombination in the said mammal.

15. A method according to any of claims 11 to 14, wherein the vitamin D is 1α,25-dihydroxyvitamin D3.

16. A transgenic rodent whose genome is heterozygous or homozygous for an engineered disruption in a rodent NuSAP gene coding for SEQ ID NO:1 or its rodent homologue, wherein said engineered disruption in a heterozygous state inhibits overall or tissue specific production of a NuSAP protein having the amino acid sequence SEQ ID NO:2 or the rodent homologue thereof, thereby resulting in a transgenic rodent which has increased tendency, with respect to a wild-type rodent, to disorders associated with DNA replication, cell proliferation and meiosis.

17. A transgenic rodent according to claim 16, further comprising a promoter in front of the gene for modulating the expression of the protein in comparison with wild type.

18. A ribozyme capable of cleaving the mRNA of a protein according to claim 2, the said ribozyme including sequences complementary to portions of mRNA obtained from a eukaryotic polynucleotide according to claim 1.

19. An antisense oligonucleotide capable of blocking expression of a eukaryotic polynucleotide according to claim 1.

20. A vector which produces in a cell, other than a human germ line cell, the ribozyme of claim 18 or the antisense oligonucleotide of claim 19.

21. A monoclonal antibody being able to discriminate between a Nuclear Spindle-Associating Protein according to claim 2 with and without a post-translationally modified amino-acid.

22. A method of prevention or treatment of a disorder associated with DNA replication, cell proliferation or meiosis, comprising administering to a mammal suffering from such disorder a therapeutically effective amount of a eukaryotic polynucleotide according to claim 1.

23. A method of prevention or treatment according to claim 22, wherein the said disorder is cancer.

24. A method of prevention or treatment according to claim 22 or claim 23, wherein the said disorder is a cancer of a tissue selected from the group consisting of kidney, stomach, small intestine, lung, ovary, cervix, breast and uterus.

25. A method of prevention or treatment according to claim 22, wherein the said disorder is a disorder of reproduction in eukaryotic cells.

26. A method of prevention or treatment according to claim 22, wherein the said disorder is a disorder of the immune system.

27. A method of prevention or treatment according to claim 22, wherein the said disorder is a disorder related to bones of mammals.

28. A method of prevention or treatment according to claim 22, wherein the said disorder is a disorder caused by mutations in nuclear structural proteins, such as the Emery-Dreifuss muscular distrophy, the dilated cardiomyopathy or lipodistrophy.


SEQUENCE LISTING (4 sequences)

SEQ ID NO: 1 - DNA, 2136 bp, Mus musculus
  CDS (71)..(1354): putative  SAP domain AA 9-43  , putative NLS AA 203-220
ccagttttgagccgctctgttttgagttgtgtggctgtttttctgggaatcaccgagattgcagaacgcgatgaccgtcccctctgcagaggagctggactcctttaagtacagcgacctgcaaaatttagccaaaaggctgggcctccgggctaacatgaaggcagacaagttgttaaaagccttgaaagcacacctgaatccagaaacaaggaaagaaaataaaaatcaggatgaaaatcaattctccactgatgaaactgagatacacgttagcagtgaggagcaagctgagacggaatcaggtggtcacgtcaccaaaacgaggaggaggaggaggaagaagcacaagaccatccatggaattcctacctcccagactttgttgcaggatcatttggagatgaaaggaactgatagtagtaacttccaaaatcaagaaaatcaggaaaatcaagaccccagggatacagcagaagttccttctctgccggagcagaggccagaggacggcaatgcggcttcttcaggagaaggagaagtaaatgacattaaagattcaaagaagcctttagaaaaaagatctctatgcacggatgagttttctaaacttgggaacaataaaaggacttcagccacaacaccaaactttaagaagcttcatgaggctcgttttaagaaaatggaatccattgatgaatatattatgaggaaaaagaaacaccttaaagaacacagttcacttaatgaactaaagcttgacaaaaaagggatagtgaccccagttcctccaagaggaaggctctctgtaccctgtactcctgccaggcagcagtgcccacaaggccactcagcaactaaaatgaatgtcaggttttcagctgctactaaagacaatgaacataagtgctcactgaccaagacaccagccagaaagtctccacacgtgactgcacctgggagtgcttcaaaaggccaggctgtgttcaggacacccaagtcaaaggccactgaaaggacttctattgcagttattacccctttcaagttgatgactgaagcaacacagactccaagttctagtaagaagccagtatttgatctcaaagcaagcttgtctcgtcccctcaactacaagccacacaaaggagagctgaaaccttggggacaagctaaagagaacaattctctgaacgaacgtgtaagcagagttaccttccacaggaaaacttacaaacaacctcatctccaaaccagggaagaacgatggaagagacaagagcaagaacgaaaggagaagaaagaaaagcttttggaagctcgaagaaacctgggtgtgactaaagcccagtgaccctgtctgttcctttactctaacttgttttccttttgtatgttttttactctttctctacttcagtcaaaagctcttttctatcataacttttggtcataatttgtgtagtgtctcttctgtgctatatctgagaatatattatcaccttaaagttcataactaaagtagttttcatctaatgccctatacatctccaatttttaaaagacttgtcctcatgattcttaacagaggtttttcatgtcaaggtcctgatagttccgagggagatgacctgcacccttttctgcacaagctgtagcagaatcttagcatgagaaaataagatatcgctcataggaaaagggaggctggaagaccacatttttgttcagtagcctggaaaatctattagtcttattgaaatcttattttatcaaggtaaaaagttttagcttataagagccttgttctgacttttcatgtatttgccatctgtcattcattataatcctaaacgagaaaacctctactctctctacttgttaaataaaagccacagggcaaagggtgtgctttagttgtacaacacttgtctatcacactcctgtccctgaactgtaccaccaaaaccaaagctgagaattgctgctgtaagaattactgtcattggcagacttttttcttacaagtagtaaagagaggaaagctgcaaggggtgaccttctgatctttgctctgccttacagagattctgagatgtgtgtaatgcttacaatgttcataaataaaaattttca

SEQ ID NO: 2 - protein, 427 aa, Mus musculus
MTVPSAEELDSFKYSDLQNLAKRLGLRANMKADKLLKALKAHLNPETRKENKNQDENQFSTDETEIHVSSEEQAETESGGHVTKTRRRRRKKHKTIHGIPTSQTLLQDHLEMKGTDSSNFQNQENQENQDPRDTAEVPSLPEQRPEDGNAASSGEGEVNDIKDSKKPLEKRSLCTDEFSKLGNNKRTSATTPNFKKLHEARFKKMESIDEYIMRKKKHLKEHSSLNELKLDKKGIVTPVPPRGRLSVPCTPARQQCPQGHSATKMNVRFSAATKDNEHKCSLTKTPARKSPHVTAPGSASKGQAVFRTPKSKATERTSIAVITPFKLMTEATQTPSSSKKPVFDLKASLSRPLNYKPHKGELKPWGQAKENNSLNERVSRVTFHRKTYKQPHLQTREERWKRQEQERKEKKEKLLEARRNLGVTKAQ

SEQ ID NO: 3 - DNA, 1551 bp, Homo sapiens
  CDS (69)..(1391): putative SAP domain AA 9-43 ; putative NLS 194-211
gggatttgaaccncgctgacgaagtttggtgatccatcttccgagtatcgccgggatttcgaatcgcgatgatcatcccctctctagaggagctggactccctcaagtacagtgacctgcagaacttagccaagagtctgggtctccgggccaacctgagggcaaccaagttgttaaaagccttgaaaggctacattaaacatgaggcaagaaagggaaatgagaatcaggatgaaagtcaaacttctgcatcctcttgtgatgagactgagatacagatcagcaaccaggaagaagctgagagacagccacttggccatgtcaccaaaacaaggagaaggtgcaagactgtccgtgtggaccctgactcacagcagaatcattcagagataaaaataagtaatcccactgaattccagaatcatgaaaagcaggaaagccaggatctcagagctactgcaaaagttccttctccaccagacgagcaccaagaagctgagaatgctgtttcctcaggtaacagagattcaaaggtaccttcagaaggaaagaaatctctctacacagatgagtcatccaaacctggaaaaaataaaagaactgcaatcactactccaaactttaagaagcttcatgaagctcattttaaggaaatggagtccattgatcaatatattgagagaaaaaagaaacattttgaagaacacaattccatgaatgaactgaagcagcccatcaataagggaggggtcaggactccagtacctccaagaggaagactctctgtggcttctactcccatcagccaacgacgctcgcaaggccggtcttgtggccctgcaagtcagagtaccttgggtctgaaggggtcactcaagccgtctgctatctctgcagctaaaacgggtgtcaggttttcagctgctactaaagataatgagcataagcgttcactgaccaagactccagccagaaagtctgcacatgtgaccgtgtctgggggcaccccaaaaggcgaggctgtgcttgggacacacaaattaaagaccatcacggggaattctgctgctgttattaccccattcaagttgacaactgaggcaacgcagactccagtctccaataagaaaccagtgtttgatcttaaagcaagtttgtctcgtcccctcaactatgaaccacacaaaggaaagctaaaaccatgggggcaatctaaagaaaataattatctaaatcaacatgtcaacagaattaacttctacaagaaaacttacaaacaaccccatctccagacaaaggaagagcaacggaagaaacgcgagcaagaacgaaaggagaagaaagcaaaggttttgggaatgcgaaggggcctcattttggctgaagattaataattttttaacatcttgtaaatattcctgtattctcaacttttttccttttgtaaatttttttttttgctgtcatccccactttagtcacgagatctttttctgctaactgttcatagtctgtgtagtgtccatgggttcttcatgtgctatgatctct

SEQ ID NO: 4 - protein, 440 aa, Homo sapiens
MIIPSLEELDSLKYSDLQNLAKSLGLRANLRATKLLKALKGYIKHEARKGNENQDESQTSASSCDETEIQISNQEEAERQPLGHVTKTRRRCKTVRVDPDSQQNHSEIKISNPTEFQNHEKQESQDLRATAKVPSPPDEHQEAENAVSSGNRDSKVPSEGKKSLYTDESSKPGKNKRTAITTPNFKKLHEAHFKEMESIDQYIERKKKHFEEHNSMNELKQPINKGGVRTPVPPRGRLSVASTPISQRRSQGRSCGPASQSTLGLKGSLKPSAISAAKTGVRFSAATKDNEHKRSLTKTPARKSAHVTVSGGTPKGEAVLGTHKLKTITGNSAAVITPFKLTTEATQTPVSNKKPVFDLKASLSRPLNYEPHKGKLKPWGQSKENNYLNQHVNRINFYKKTYKQPHLQTKEEQRKKREQERKEKKAKVLGMRRGLILAED